(19) United States
(12) Patent Application Publication
(10) Pub. No.: US 2009/0068169 A1
(43) Pub. Date: Mar. 12, 2009

(54) 2,4,6-triamino-s-triazine-based compounds which bind to the tail (fc) portion of immunoglobulins and their use

(76) Inventors: Christopher Penney, Pierrefonds (CA); Boulos Zacharie, Laval (CA); Shaun D. Abbott, Pointe-Claire (CA); Jean-Francois Bienvenu, Laval (CA); Alan D. Cameron, Montreal (CA); Jean-Simon Duceppe, St-Colomban (CA); Abdallah Ezzitouni, Laval (CA); Daniel Fortin, Rosemere (CA); Karine Houde, Montreal (CA); Nancie Moreau, Laval (CA); Nicole Wilb, Laval (CA); Brigitte Grouix, Montreal (CA); Lyne Gagnon, Laval (CA)

Correspondence Address: NIXON & VANDERHYE, PC, 901 NORTH GLEBE ROAD, 11TH FLOOR, ARLINGTON, VA, 22203, US

(21) Appl. No.: 11/661,233

(22) PCT Filed: Sep. 2, 2005
(86) PCT No.: PCT/CA05/01344

§ 371 Date: Sep. 11, 2008

Related U.S. Application Data
(60) Provisional application No. 60/606,909, filed on Sep. 3, 2004.

(51) Int. Cl. A61K 31/53; C07D 251/54; A61K 38/00; A61K 39/395; A61K 35/14; A61K 35/00; C07K 1/14

(52) U.S. Cl. 424/130.1; 544/197; 514/245; 514/2; 424/529; 424/520; 530/412

(57) ABSTRACT

The present invention describes new compounds which are useful for binding to the tail or Fc portion of immunoglobulins and so have utility in those applications which require the non-covalent binding interaction of a molecule with the Fc portion of immunoglobulins. Such applications include the detection and purification of immunoglobulins as well as the treatment of certain autoimmune diseases.

### CROSS-REFERENCE TO RELATED APPLICATIONS

This application claims the benefit of provisional U.S. Appln. No. 60/606,909, filed Sep. 3, 2004; the contents of which are incorporated by reference herein.

### FIELD OF THE INVENTION

The present invention comprises new compounds described by the following general formula (formula I):

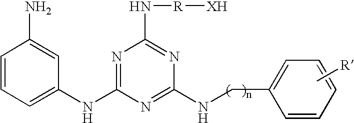

where R is a straight chain or cyclic alkyl group, X is oxygen or sulfur or an imino group or is absent, R′ is an amino or methoxy group or fluorine or chlorine atom and n is 0, 1 or 2. Alternatively, —R—XH may be replaced by

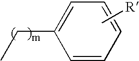

wherein R′ is defined as above and m=1 or 2, or —R—XH may be replaced by a hydrogen atom. These compounds are useful in that they bind to the tail or Fc portion of immunoglobulins and so have utility in those applications which require the non-covalent binding interaction of a molecule with the Fc portion of immunoglobulins. Such applications include the detection and purification of immunoglobulins as well as the treatment of certain autoimmune diseases.

### BACKGROUND OF THE INVENTION

Monoclonal antibodies represent the fastest growing segment of the prescription drug market. Over one hundred recombinant antibodies are currently in clinical trials targeted towards the treatment of cancer, autoimmune and infectious diseases. Other uses of monoclonal antibodies include diagnostic and imaging applications. As a compound class, therapeutic monoclonal antibodies offer important advantages. For example, they are highly specific for their molecular or biochemical targets and they tend to be stable in serum or exhibit a long half-life. However, therapeutic monoclonal antibodies are difficult to manufacture and subsequently expensive to produce. An important issue is the limited number of purification techniques available. Antibodies are generally purified by classical (e.g., ion-exchange) column or batch chromatography or by affinity chromatography with bacterial protein A or protein G covalently attached to a solid-phase support. The heavy reliance on bacterial protein A is reflected by the fact that the demand for protein A resin is approximately 10,000 liters a year. Furthermore, this demand is projected to increase at a rate of approximately 50% per year. However, protein A is toxic (e.g., pyrogenic) and there is always a concern that a small amount of protein A will be released or leach from the column into the purified antibody targeted for human use. There is clearly a need for novel, safe, synthetic low molecular weight (non-protein) compounds which can selectively be used to bind to immunoglobulins and subsequently expedite their purification.

U.S. Pat. No. 6,117,996 (2000) describes triazine based synthetic affinity ligands covalently attached to a solid-phase support as potential replacements for protein A affinity columns. Although this patent does not provide specific exemplification of purification of monoclonal antibodies, it nonetheless discloses the potential of triazine based compounds linked to a solid-phase support for the purification of many proteins of therapeutic importance. Important to note is the fact that these triazine based compounds are covalently linked to a polysaccharide support. Indeed, the scope of the patent is limited to novel affinity ligand-matrix conjugates wherein the triazine based compound covalently attached to the solid-phase support constitutes the invention. This patent is supported by a number of publications which disclose the use of triazine based compounds covalently attached to agarose for the purification of IgG. For example, S. F. Teng et al., Journal of Chromatography 740, 1-15 (2000); S. F. Teng et al., Journal of Molecular Recognition 12, 67-75 (1999) and R. Li et al., Nature Biotechnology 16, 190-195 (1998) describe IgG binding ligands of the following structure:

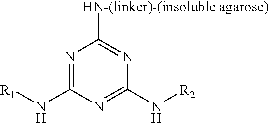

In these publications, the most effective or preferred IgG binding ligands are those where R₁=naphthol and R₂=phenol or R₁=phenyl and R₂=hydroxyphenethyl. In all cases, patent or scientific paper, the triazine based compound is covalently attached to an insoluble solid-phase (agarose) support. In fact, the ability of the triazine based compounds to bind to immunoglobulin is determined in a solid-phase binding assay. These compounds, in solution phase or not attached to a solid-phase support, bind only weakly to immunoglobulin. For example, the Nature Biotechnology paper cited above reveals that when R₁ is phenyl, R₂ is hydroxyphenethyl and (linker-agarose) is replaced with an aminoethyl group, the resultant soluble triazine based compound binds to the tail portion of IgG with approximately one-thousandth of the affinity exhibited between bacterial protein A and IgG.

WO 98/08603, published Mar. 5, 1998, also describes low molecular weight synthetic (primarily substituted benzoic acids) affinity ligands covalently attached to a solid-phase support as potential replacements for protein A affinity columns. Once again, the scope of the patent is limited to solid-phase matrices, preferably epichlorohydrin activated agarose, functionalized with mono- or bicyclic aromatic or heteroaromatic ligands. In this case, the invention does not include triazine based compounds. However, as noted with the triazine based compounds described above, these compounds, in solution phase or not attached to a solid-phase support, bind only weakly to immunoglobulins.

Most recently, WO 2004/035199 A1, published Apr. 29, 2004, describes triazine based synthetic affinity ligands covalently attached to a solid-phase support for purification of antibodies. Again, the scope of the patent is limited to novel affinity ligand-matrix conjugates wherein the triazine based compound covalently attached to the solid-phase support constitutes the invention. The structure of the preferred affinity ligand-conjugate is as follows:

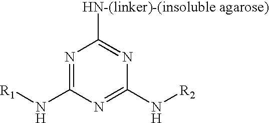

where R₁=benzamide and R₂=carboxypropyl or R₁=phenethyl and R₂=2-hydroxypropyl. Additionally, these affinity ligand-matrix conjugates preferentially bind to the antigen binding portion, or Fab fragment, of antibodies and are thus distinct from compounds of the present invention in that the latter bind to the tail or Fc portion of antibodies.

Once again, the important point which distinguishes the above cited prior art from this invention is that compounds of the present invention bind with high affinity to antibodies either in solution or attached to a solid-phase support. The molecular feature present in the compounds described in this invention, but not present in prior art citations, which appears responsible for this high affinity interaction with antibodies is an aryl or anilino amine grouping.

Peptides and polypeptides or small proteins have also been described in the literature which bind to the tail portion of IgG and so mimic the behavior of bacterial protein A. For example, G. Fassina et al., Journal of Molecular Recognition 9, 564-569 (1996) and U.S. Pat. No. 5,880,259 (1999) describe a tetrameric presentation of a tripeptide sequence (Tyr Thr Arg) which could be used to purify IgG after covalent attachment to a solid-phase support. Although quantitative data was not reported, this peptide appears to bind IgG in solution as evidenced by the fact that it was discovered, unlike the prior art cited above, in a solution assay. However, this tetrameric tripeptide is a much larger molecule than the synthetic compounds described above. Even larger polypeptide ligands, or protein A mimics, include a histidine-tagged fragment of protein A disclosed by C. P. Johnson et al., Bioconjugate Chemistry 14, 974-978 (2003).

As can be seen from the above, small synthetic compounds covalently attached to a solid-phase support or larger peptides or polypeptides which mimic bacterial protein A can be used to bind immunoglobulins. However, the literature does not disclose small synthetic compounds which can bind with high affinity to immunoglobulins in solution. That is, compounds which are equipotent with bacterial protein A as regards their ability to bind to the tail or Fc portion of IgG immunoglobulin. It is therefore an objective of the present invention to provide novel compounds which effectively bind, in solution or attached to a solid-phase, to antibodies.

### SUMMARY OF THE INVENTION

The present invention satisfies the need for novel low molecular weight (<500 kD) synthetic compounds which can effectively bind to immunoglobulins either in a solution phase or as part of a solid-phase after covalent attachment to an insoluble matrix. Such compounds have utility in that resulting from this invention is a method for purification of IgG immunoglobulin when the compounds of this invention are covalently attached to an insoluble solid-phase support. Such compounds also have further utility in that resulting from this invention is a method for the treatment of chronic autoimmune disease wherein the etiology and progression of the disease is attributed to, at least in part, immune complexes along with antibodies directed to self or so-called autoantibodies.

Only now is it being recognized that inflammation caused by immune complexes in the joints (arthritis), the kidneys (glomerulonephritis) and blood vessels (vasculitis) is a major cause of morbidity in autoimmune disease as noted by P. M. Hogarth et al., Annual Reports in Medicinal Chemistry 37, 217-224 (2002). Increased immune complex formation correlates with the presence of autoantibodies and the latter can also contribute to tissue inflammation. In some autoimmune diseases, the presence of autoantibody contributes significantly to disease pathology. This has been clearly demonstrated, for example, in systemic lupus erythematosus (SLE; anti-DNA antibodies), immune thrombocytopenic purpura (ITP; antibody response directed to platelets) and to a lesser extent rheumatoid arthritis (IgG reactive rheumatoid factor). The importance of the role of immune complexes and free autoantibodies is further demonstrated by the fact that successful treatment of certain autoimmune diseases has been achieved by the removal of immune complexes and free antibody by means of specific immunoadsorption procedures. For example, the use of an apheresis procedure in which immune complexes and antibodies are removed by passage of a patient's blood through an immunoaffinity (PROSORBA®) column was approved by the U.S. Food and Drug Administration in 1987 for ITP and in 1999 for rheumatoid arthritis. However, currently there is no approved method for the treatment of autoimmune diseases which facilitates the elimination of immune complexes and autoantibodies by administration of a drug.

Therefore, in accordance with this invention, certain triazine based compounds which bind effectively in solution to the tail portion of immunoglobulins (either as part of immune complexes or as free autoantibodies) can be administered to a mammal, preferably a human, in need of such treatment for autoimmune disease. Such compounds and their pharmaceutical compositions are provided which are able to facilitate the clearance of immune complexes or to limit their deposition within body organs such as the kidneys. In the case where the triazine based compounds influence the elimination of immune complexes or prevent their deposition, or influence directly autoantibodies by binding to the tail or Fc portion, such compounds are expected to be particularly useful for the treatment of autoimmune diseases such as arthritis, SLE, ITP, glomerulonephritis and vasculitis.

Further aspects of the invention will be apparent to a person skilled in the art from the following descriptions and claims and generalizations thereto.

### BRIEF DESCRIPTION OF THE DRAWINGS

FIG. 1 shows the effect of compounds 1, 3 and 10 on the first challenge of DTH.

FIG. 2 shows the effect of compounds 1, 3 and 10 on the second challenge of DTH.

FIG. 3 shows the effect of compounds 1 and 3 on adjuvant-induced arthritis.

FIG. 4 shows the effect of compound 35 on adjuvant-induced arthritis.

FIG. 5 shows the ability of exemplified gels to bind and elute human IgG in the presence of PLURONIC® F-68. Values are expressed as a percentage of the IgG elution fraction in the absence of PLURONIC® F-68.

FIG. 6 shows SDS-PAGE analysis of fractions from purification of mouse monoclonal antibodies from harvested cell culture fluid containing PLURONIC® F-68 with: prestained SDS-PAGE standard broad range (lane 1); monoclonal initial fraction (lane 2); flow through, spin column from Example 49-3 (lane 3); eluent at pH 3, spin column from Example 49-3 (lane 4); flow through, control (lane 5); eluent at pH 3, control (lane 6); flow through, spin column from Example 49-15 (lane 7); and eluent at pH 3, spin column from Example 49-15 (lane 8).

### DETAILED DESCRIPTION OF CERTAIN EMBODIMENTS OF THE INVENTION

The present invention includes compounds, or pharmaceutically acceptable derivatives thereof, of the following general formula:

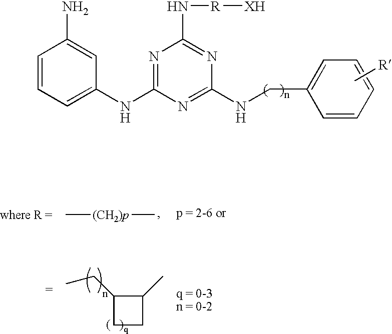

### X=NH, O, or S

### and R′=NH₂, OCH₃, F or Cl.

In another aspect of the present invention, the group —R—XH may be replaced by

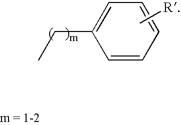

In such a case, the general formula becomes:

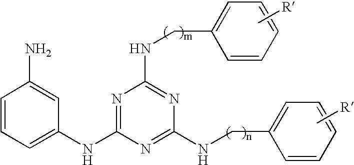

wherein R′ is defined as above but, if two R′ substituents are present in the same compound, both R′ substituents may be the same (amino, methoxy, fluorine) or one R′ substituent can be an amino group or fluorine atom while the second is a methoxy group. Also, m and n are defined as above but it is not necessary that m is equal to n.

In still another aspect of the present invention, in the case where R′ is meta amino and n=0 then —R—XH may be replaced such that the general formula is:

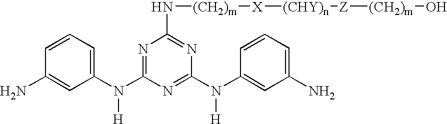

wherein m=1-2, n=2-4, X=CHY, O, S; Y=H, OH; and Z=zero, O, S.

Finally, in still another aspect of the present invention, the group —R—XH may be replaced by a hydrogen atom. In such a case, the general formula becomes:

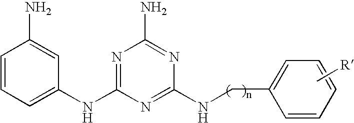

wherein R′ and n are defined as above.

When m is equal to n and R′ is an amino or methoxy group or fluorine atom, then the compound becomes a bis(alkaryl) substituted triazine (m=n=1 or 2). However, this symmetric substitution does not represent a preferred aspect of this invention. A preferred embodiment of this invention is provided by the bis(aryl) substituted triazine that results when n=0 and the corresponding R′=meta NIH2. The latter is less susceptible to oxidation.

Regardless of the structure defined above, it is a preferred embodiment of this invention that R′ is an amino group. Most preferred is that the amino group is located at the meta position. Least preferred is that the amino group is located at the ortho position because of its reduced bioactivity and increased susceptibility to oxidation. Therefore, particularly preferred compounds are those represented by the following structures:

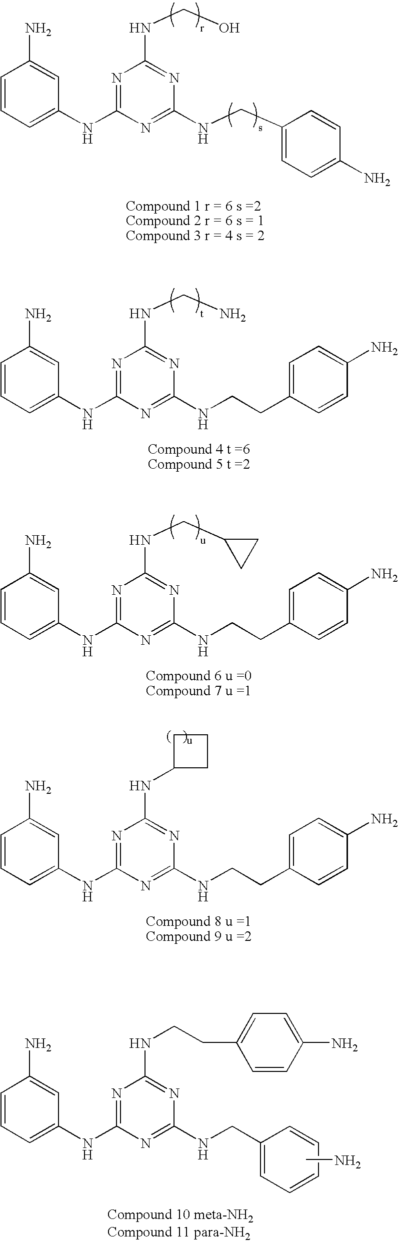

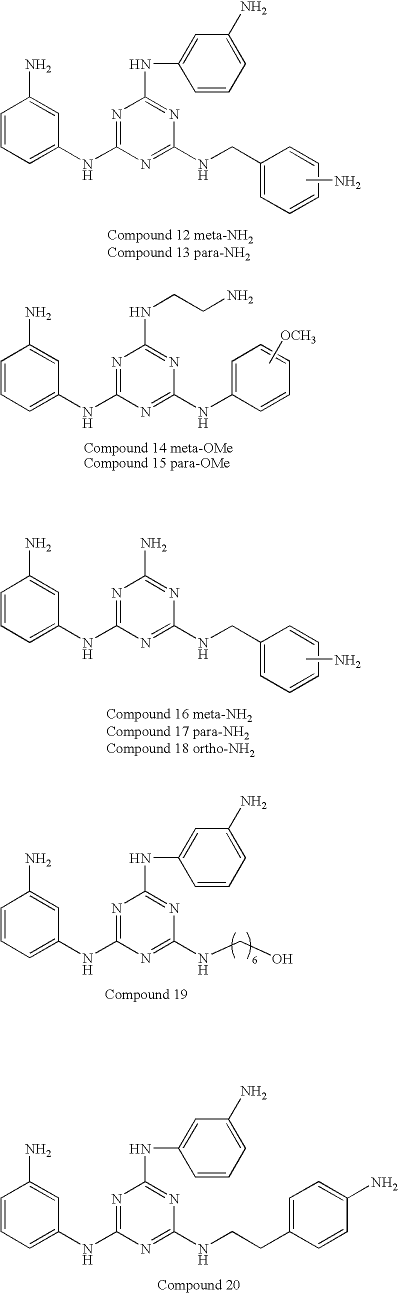

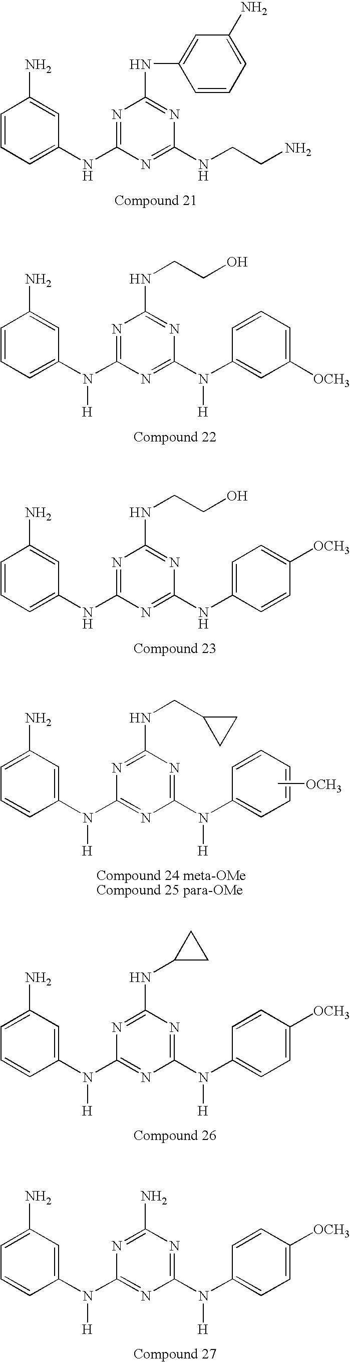

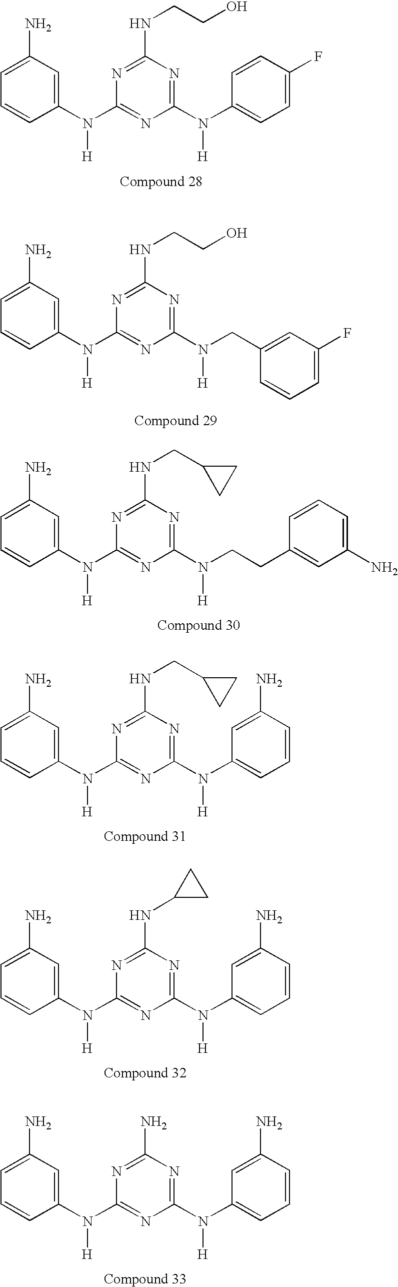

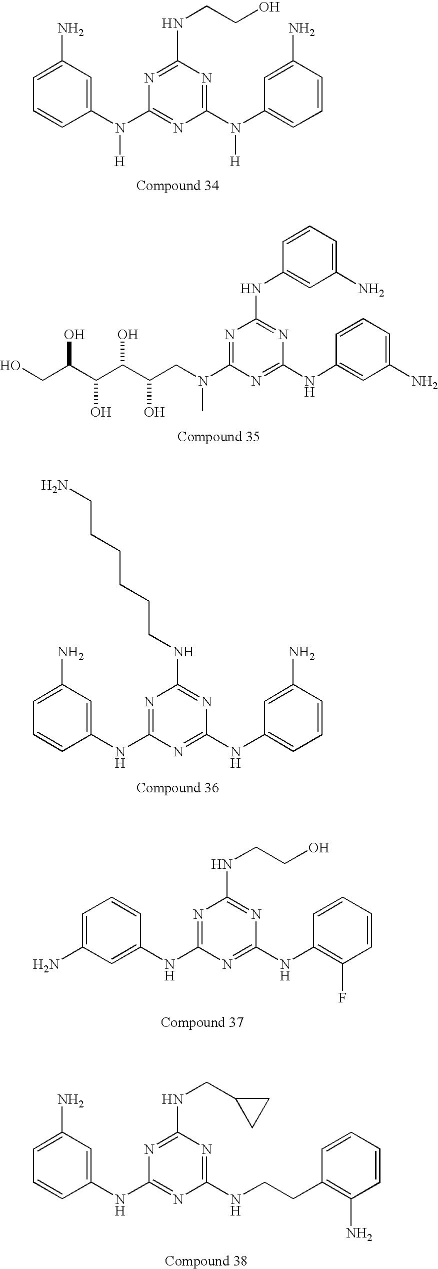

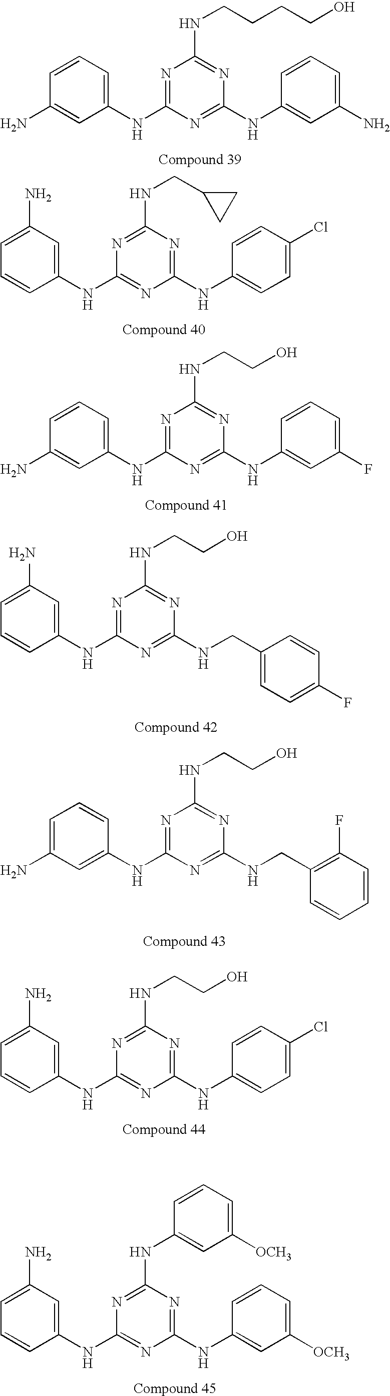

It will be appreciated by anyone skilled in the art that although the scope and subsequent claims of the invention are limited to the general formula defined above, any minor modification of that formula such as substitution with two amino or two methoxy groups, instead of one as described in formula I, constitutes an obvious modification of this invention. Similarly, modification of one or more amino groups by acylation or alkyl sulfonylation, to provide a prodrug format or alter drug activity, constitutes another obvious modification of this invention. It is noteworthy that in the case where two of the triazine substituents are the same meta-aminoanilino group, the third substituent although defined above can be almost any grouping of ten carbon atoms or less and still retain significant binding activity. The bis-aminoanilino arrangement results in such potent binding activity (for example, compounds 12 and 13 in Table 1) that almost any third substituent will be tolerated.

Compounds of the present invention may facilitate the clearance of immune complexes by phagocytosis or may limit the deposition of complexes within body organs and tissues by their ability to antagonize the binding of immune complexes to organ and tissue surfaces. The mechanism by which immune complexes attach to various surfaces can involve binding to cell surface Fc receptors. Fc receptors are glycoproteins of inflammatory leukocytes that bind the Fc (tail) portion of immunoglobulins. Fc receptors are also present on numerous tissues and provide a site for attachment and subsequent deposition of immune complexes onto tissue surfaces. For example, the deposition in kidney tissue of autoantibody containing complexes by binding to Fc receptors is thought to trigger an inflammatory response typical of SLE which can lead to glomerulonephritis. Well characterized Fc receptors include: FcγRI, FcγRII, and FcγRIII (IgG receptors); FcεRI (the IgE receptor) and FcαRI (the IgA receptor). Interestingly, Staphylococcal protein A is a cell-surface bacterial protein which can bind to the Fc (tail) portion of most antibodies. For example, protein A will bind to human IgG1, IgG2 and IgG4 immunoglobulins. More importantly, it has been known for many years that protein A can inhibit the binding of IgG antibody containing immune complexes to Fc receptors. For example, A. Sulica et al., Immunology 38, 173-179 (1979) reported that protein A does inhibit IgG containing immune complex binding to Fc receptors but protein A enhances binding of IgG to lymphocytes and macrophages.

More recently, with the availability of Fc receptor (γ chain) deficient mice, it became possible to establish the primary role of the IgG Fc receptors (FcγR) in mediating the effector responses seen in autoimmune diseases such as SLE and rheumatoid arthritis, as noted by M. Marino et al., Nature Biotechnology 18, 735-739 (2000). More specifically, these authors stated that agents which can interfere with the binding of immune complexes to FcγR should ameliorate SLE. They provided experimental support for this statement by treating a special strain of mice (MRL/Ipr) that develops a syndrome which is similar to human SLE with a peptide which binds to the Fc portion of IgG. The survival rate of treated animals (80%) was significantly greater than untreated animals (10%). In a recent review by P. M. Hogarth, Current Opinion in Immunology 14, 798-802 (2002), it is stated that FcγR acts early in the inflammation process and engagement by immune complexes is a potent signal for the release of proinflammatory cytokines such as TNFα. In those cases where compounds of the present invention affect some aspect of immune complex clearance or deposition, they may do so by their ability to mimic protein A. That is, such compounds can bind to the Fc portion of human IgG as ascertained by their ability to inhibit the binding of protein A to human IgG, as determined in vitro by competitive ELISA. By binding to the Fc portion of human IgG in a fashion similar to protein A, such protein A mimic compounds may disrupt the binding of IgG containing immune complexes to FcγR. Subsequently, this should prevent deposition of immune complexes and thereby facilitate their clearance as well as diminish the release of proinflammatory cytokines. Additionally, protein A mimic compounds may bind to other proteins which play a role in the acute-phase of the inflammatory response, such as C-reactive protein, and which have an immunoglobulin (antibody-like) structure.

The present invention provides novel compounds as defined by the general formula I above which are useful for the treatment of chronic autoimmune disease. As these compounds may facilitate the clearance of immune complexes by phagocytosis or may limit the deposition of immune complexes within body organs and tissues in addition to other aspects of the inflammation process, they may be particularly useful for the treatment of those autoimmune diseases where immune complexes play an important role in disease pathology. Examples of such autoimmune diseases include arthritis, SLE, ITP, glomerulonephritis, and vasculitis. Additionally, compounds of the present invention may inhibit the biosynthesis and subsequent release of proinflammatory cytokines such as TNFα by disruption of immune complex binding to FcγR on monocyte/macrophage and neutrophils.

Compounds of the present invention include all pharmaceutically acceptable derivatives, such as salts and prodrug forms thereof, and analogues as well as any geometrical isomers or enantiomers. Formulations of the active compound may be prepared so as to provide a pharmaceutical composition in a form suitable for enteral, oral (including sublingual, pulmonary and rectal), parenteral (including intramuscular, intradermal, subcutaneous and intravenous) or topical (including ointments, creams or lotions) administration. In particular, compounds of the present invention may be solubilized in an alcohol or polyol solvent (e.g., solutol HS 15 (polyethylene glycol 660 hydroxystearate from BASF), glucose, glycerol, ethanol, etc.) or any other biocompatible solvent such as dimethyl sulfoxide (DMSO) or cremophor EL (also from BASF). The formulation may, where appropriate, be conveniently presented in discrete dosage units and may be prepared by any of the methods well-known in the art of pharmaceutical formulation. All methods include the step of bringing together the active pharmaceutical ingredient with liquid carriers or finely divided solid carriers or both as the need dictates. When appropriate, the above-described formulations may be adapted so as to provide sustained release of the active pharmaceutical ingredient. Sustained release formulations well-known to the art include the use of a bolus injection, continuous infusion, biocompatible polymers or liposomes.

Suitable choices in amounts and timing of doses, formulation, and routes of administration can be made with the goals of achieving a favorable response in the mammal (i.e., efficacy), and avoiding undue toxicity or other harm thereto (i.e., safety). Therefore, “effective” refers to such choices that involve routine manipulation of conditions to achieve a desired effect: e.g., reducing or otherwise ameliorating tissue injury associated with an immune response to body constituents (organs and tissues like adrenal, eye, joint, kidney, liver, lung, pancreas, nervous system, skin, thyroid etc.); restoring the immunological status or normalizing a pathological disorder/condition of the mammal (e.g., antibody titer, immune cell subsets, signaling by cytokines or chemokines, antibody-antigen immune complexes etc.); removal of free antibodies and/or antibody-antigen immune complexes from the circulation; laboratory indicia of autoimmune disease (e.g., concentration or absolute amount of soluble mediators of inflammation, presence of autoantibodies, cellular proliferation etc.); and combinations thereof. In particular, deleterious effects of conventional anti-TNFα treatment may be avoided.

The amount of compound administered is dependent upon factors such as, for example, bioactivity and bioavailability of the compound (e.g., half-life in the body, stability, and metabolism); chemical properties of the compound (e.g., molecular weight, hydrophobicity, and solubility); route and scheduling of administration; and the like. It will also be understood that the specific dose level to be achieved for any particular patient may depend on a variety of factors, including age, health, medical history, weight, combination with one or more other drugs, and severity of disease.

The term “treatment” refers to, inter alia, reducing or alleviating one or more symptoms of autoimmune disease in a mammal (e.g., human) affected by disease or at risk for developing disease. For a given patient, improvement in a symptom, its worsening, regression, or progression may be determined by an objective or subjective measure. Treatment may also involve combination with other existing modes of treatment and agents (e.g., anti-inflammatory drugs, agents binding TNFα like antibody or soluble receptor, NSAIDs, corticosteroids, DMARDs). Thus, combination treatment may be practiced. In such embodiments, it is preferred that toxicity of chronic treatment or the additional agent is at least reduced or avoided by reducing the amount or concentration of the additional agent used in comparison to treatment without a compound of the present invention.

It will be appreciated by those skilled in the art that the reference herein to treatment extends to prophylaxis as well as therapy of established or chronic autoimmune disease. It will be further appreciated that the amount of a compound of the invention required for treatment will vary not only with the particular compound used for treatment but also with the route of administration, the nature of the autoimmune condition being treated and the age and general health of the patient. The dose to be administered will ultimately be at the discretion of the physician. In general, however, the dose will be in the range from about 0.1 mg/kg to about 200 mg/kg of body weight per day. Preferably, doses will range from about 1 mg/kg to about 100 mg/kg per day. More preferably, the range will be between about 2 mg/kg to about 50 mg/kg per day.

Finally, and where appropriate, compounds of the present invention may be used in combination with other treatments for autoimmune disease well-known to the art. Other prior art treatments include those described above as represented by nonsteroidal anti-inflammatory drugs (NSAIDs; e.g. ibuprofen, aspirin, naproxen, etodolac, and ketoprofen); corticosteroids (e.g., hydrocortisone, pregnisone, and dexamethasone); disease-modifying anti-rheumatic drugs (DMARDs; e.g. cytotoxic drugs like methotrexate or azathioprine, immunosuppressants like cyclosporin or FK506, hydrochloroquine, organogold salts) and biologicals. The individual components of such combinations may be administered either sequentially or simultaneously in separate or combined pharmaceutical formulations. Alternatively, new pharmaceutical formulations may be created to accommodate the combination of compounds of this invention with conventional treatments for autoimmune disease.

Compounds of the present invention may also be used as affinity agents to bind antibody (e.g., human isotypes like IgM, IgD, IgA1, IgA2, IgE, IgG1, IgG2, IgG3, and/or IgG4). Free (i.e., not bound to antigen) antibody and/or antibody-antigen immune complex may be specifically bound by such affinity agents. Additionally, antibody-like proteins or fusion proteins which consist of Fc immunoglobulin domains or tails fused or covalently linked with other proteins or peptides may also be specifically bound by such affinity agents. Large affinity complexes may be isolated by selective precipitation or differential centrifugation, or identified by flocculation assays. But it is preferred to immobilize one or more compounds to an insoluble support material (e.g., agarose, dextran, cellulose, polyacrylamide, other polymeric materials, silica, and glass) preferably covalently linked directly or indirectly by a linker. For example, immobilization may occur through biotin-streptavidin interaction. A compound of the present invention may be synthesized in situ on the support or through an activated organic linker. Optionally, the linker may be cleavable (e.g., by a reducing agent or site-specific protease) such that the compound (with or without bound antibody) may be detached from the support. For example, one or more compounds of the present invention may be covalently linked to a support in the form of a glass slide, multiwell plate, optical fiber, protein chip or test tube for assays and analysis; tissue culture dish for incubating cells or antigen; and magnetic beads, porous membrane or chromatographic media for separation. Antibody or other Fc containing material may be bound to one or more compounds of the present invention (i.e., isolation), and then optionally separated from unbound material (with or without washing and multiple rounds of binding under different conditions) to purify Fc containing material. For example, ionic strength (e.g., salt concentration) or pH may change binding conditions and be used to release Fc containing material. This is illustrated in the accompanying examples wherein human immunoglobulin is bound to compounds attached to a solid-phase support at neutral pH and then eluted at acidic pH. An important aspect of this invention is the fact that antibodies can be bound to one or more compounds of the present invention, while attached to a solid-phase support, in the presence of 0.1% PLURONIC® F-68. PLURONIC® F-68 is a non-ionic detergent which is used in cell culture for the production of monoclonal antibodies. This detergent functions to protect cells from hydrodynamic damage but it also inhibits the binding of antibodies to most prior art affinity columns.

Free antibody and/or immune complexes may be isolated for clinical laboratory diagnosis. Apheresis using standard or fluidized bed chromatography may be used to remove free antibody and/or immune complexes from the circulation: a physiological fluid (e.g., blood) is incubated with insoluble support material (e.g. derivatized silica) on which one or more compounds of the present invention are attached, at least some antibody material is bound to the compound(s) and immobilized on the support, bound antibody is separated from the rest of the physiological fluid, and at least some of the remaining (soluble) components of the physiological fluid is returned to the mammal from whom it was obtained. It is convenient to package the device containing one or more compounds of the invention for apheresis (e.g., a column) under aseptic conditions and to replace it after every use.

Antibody may be isolated from a composition and then optionally separated to any desired degree of purification. An antibody containing composition is incubated with insoluble support material on which one or more compounds of the present invention are attached, and at least some antibody material is bound to the compound(s) and immobilized on the support. Bound antibody may be separated from the remainder of the composition and that remainder is depleted of total antibody or that fraction of antibody which binds (e.g., one or more isotypes). Isolated antibody on the support may be released by washing or cleaving the linker. Either enriched antibody or the components of the depleted composition or both is the desired product. It is convenient to repeat binding and washing under different incubation conditions to increase the efficiency of isolation and separation.

Therefore, in another embodiment of the present invention, a device or kit is provided for use in the methods described above. For example, it may be used to bind antibody, for isolation of antibody, to remove antibody from a composition or the circulation, for separation of antibody, and to purify antibody from a source material or other composition. The product may be packaged aseptically under pharmaceutically acceptable conditions or stored under sterile conditions for the clinical laboratory. One or more compounds of the present invention are attached to an insoluble support material and packaged in a device (e.g., column) or kit with one or more optional components: storage buffer, binding and washing solutions, and an agent to cleave compounds from the support. Attachment of the compound to the insoluble support is preferably achieved by covalent bonding, either directly to the support or by means of a linker, but can also be attained by non-covalent absorption of the compound onto the support material.

### EXAMPLES

The following examples further illustrate the practice of this invention but are not intended to be limiting thereof.

The general synthetic sequence for preparation of the compounds useful in the present invention is outlined in route 1 or route 2 of scheme 1. Route 1 illustrates the reaction of cyanuric chloride with monoprotected 1,3-phenylenediamine to give the dichlorotriazine intermediate. Aryl or aralkylamines were then added followed by alkylamines. Route 2 demonstrates the preparation of the dichlorotriazine intermediate as in route 1 followed firstly by the reaction with alkylamines then by the addition of aryl or aralkylamines. The last step was the removal of the protecting groups.

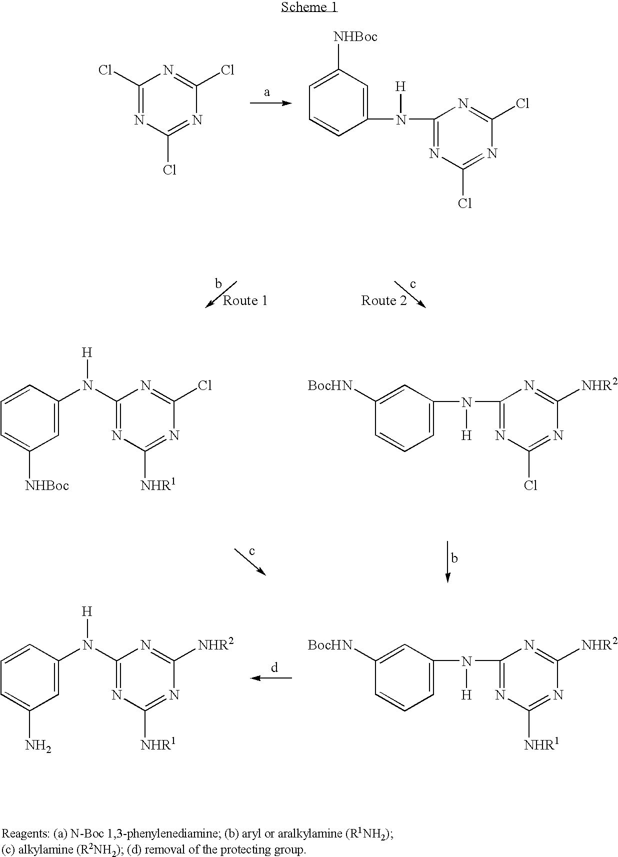

### Instrumentation

All HPLC chromatograms and mass spectra were recorded on a HP 1100 LC-MS Agilent instrument using a diode array detector. An analytical C18 column (75×4.6 mm, 5 microns) with a gradient of 10-99% acetonitrile-water containing 0.01% TFA in 5 min and a flow rate of 1 mL/min (method 1) or an analytical C18 column (75×4.6 mm, 5 microns) with a gradient of 10-40% acetonitrile-water containing 0.01% TFA in 5 min and a flow rate of 1 mL/min (method 2) or an analytical C18 column (75×4.6 mm, 5 microns) with a gradient of 0.1-20% acetonitrile-water containing 0.01% TFA in 5 min and a flow rate of 1 mL/min (method 3).

### Example 1

### Synthesis of Compound 6 (Representative Example of Route 1)

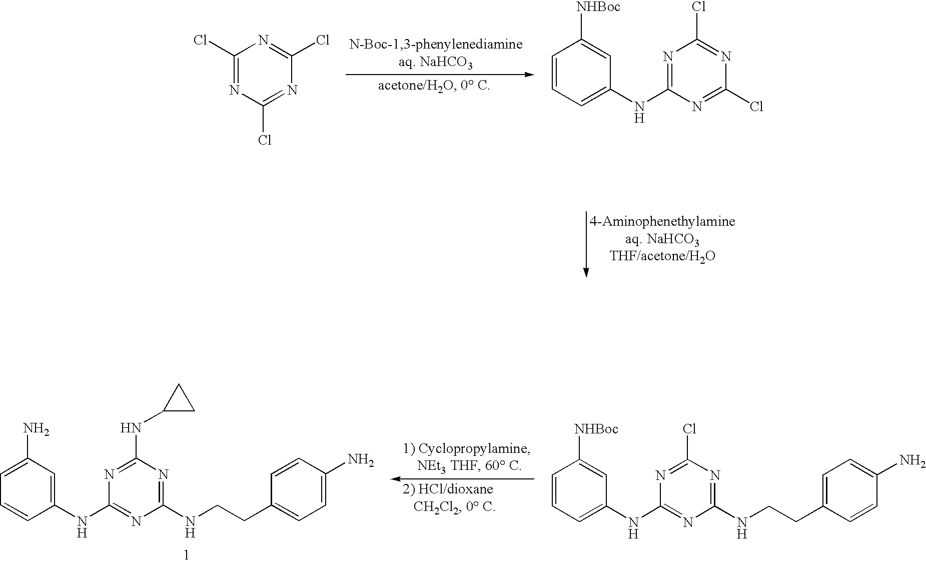

To a suspension of cyanuric chloride (2.2 g, 11.7 mmol) in acetone (15 mL) and ice (56 mL) at 0° C. was added dropwise a solution of N-Boc-1,3-phenylenediamine (2.4 g, 11.6 mmol) in acetone (7 mL). At the end of the reaction, the pH of the solution was adjusted from 1 to 7 with 5% aqueous sodium bicarbonate (25 mL). The precipitate was filtered, washed several times with water, and dried in vacuo. This gave 2,4-dichloro-6-(3-N-Boc-amino)-phenylamino-1,3,5-triazine as a white solid: 4.1 g, 99% yield; LRMS (ESI): m/z 356 (MH+), 378 (M+Na); HPLC (method 1: 8.8 min). The product was used in the next step without further purification. This triazine derivative (0.4 g, 1.2 mmol) was dissolved in THF (52 mL), acetone (13 mL), and water (13 mL) at room temperature. To this solution was added 4-aminophenethylamine (0.2 g, 1.3 mmol), followed by 5% aqueous sodium bicarbonate (5 mL). After 20 h at room temperature, the solution was diluted with water (20 mL) and ethyl acetate (20 mL). The aqueous layer was extracted with ethyl acetate (20 mL), the organic layers were washed with brine (40 mL), dried over anhydrous sodium sulfate, filtered, and evaporated to dryness. The crude residue was purified on a BIOTAGE® 40M column (silica, hexane/ethyl acetate 9:1 to 4:5) to yield 2-(2-[4-aminophenyl]ethylamino)-4-(3-N-Boc-amino)-phenylamino-6-chloro-1,3,5-triazine as a light yellow solid (0.4 g, 64%; LRMS (ESI): m/z 456 (MH+), 478 (M+Na); HPLC (method 1): 2.6 min. To a solution of this compound (48 mg, 0.1 mmol) in THF (1 mL) at room temperature was added cyclopropylamine (22 μL, 0.3 mmol), followed by triethylamine (44 μL, 0.3 mmol). After 20 h at 60° C. in a sealed tube, the solution was diluted with methanol (2 mL) and concentrated under reduced pressure. The crude residue was purified on a BIOTAGE® 12M column (silica, hexane/ethyl acetate 9:1 to 1:4) to yield 2-(2-[4-aminophenyl]ethylamino)-4-(3-N-Boc-amino)-phenylamino-6-cyclopropylamino-1,3,5-triazine as a white solid: 39 mg, 78%; LRMS (ESI: m/z 477 (MH+), 499 (M+Na); HPLC (method 1: 1.9 min). The protected triazine derivative (39 mg, 82 μmol) was dissolved in dichloromethane (0.7 mL) at 0° C. To this mixture was added 4 N hydrochloric acid in 1,4-dioxane (2.1 mL, 8.2 mmol). The reaction was stirred for 4 h at room temperature and then evaporated to dryness to yield compound 6 as a white solid. Yield of product: 31 mg, 99%; ¹H NMR (400 MHz, CD₃OD): δ 7.82-7.17 (m, 8H), 3.81 (m, 2H), 3.04 (m, 2H), 0.95 (m, 2H), 0.74 (m, 2H); LRMS (ESI): m/z 377 (MH+), 399 (M+Na); HPLC: (method 1): 0.6 min.

### Example 2

### Synthesis of Compound 9 (Representative Example of Route 2)

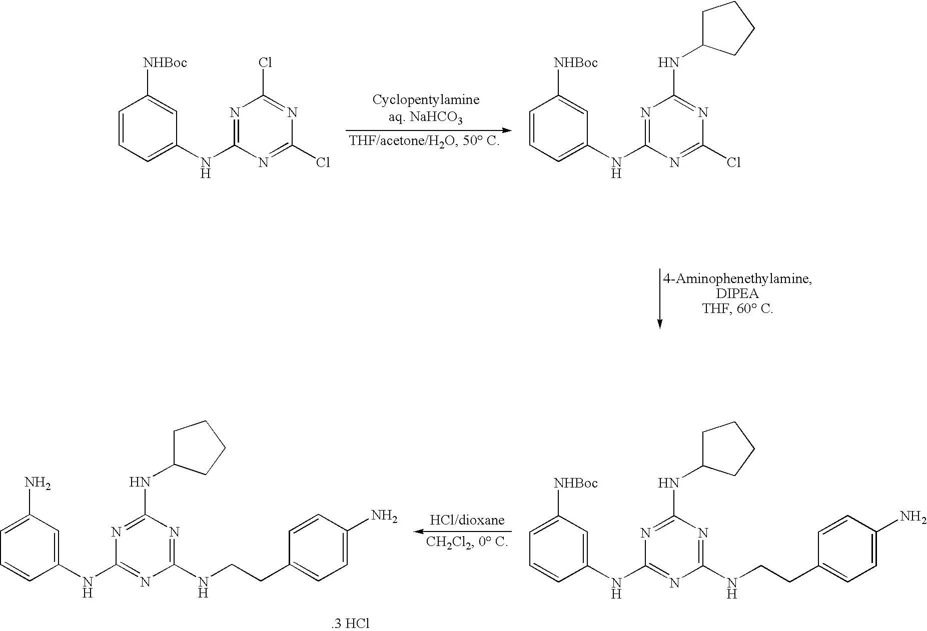

To a solution of 2,4-dichloro-6-(3-[N-Boc-aminophenyl]-amino)-1,3,5-triazine (5 g, 14 mmol) in a mixture of THF (70 mL), acetone (27 mL), and water (9 mL) at room temperature was added cyclopropylamine (1.4 mL, 14 mmol). This was followed by 5% aqueous sodium bicarbonate until the pH was 8. After 20 h at 50° C., the THF was evaporated and the solution was diluted with ethyl acetate (100 mL). The aqueous layer was extracted with ethyl acetate (200 mL), the organic layers were washed with brine (300 mL), dried over anhydrous sodium sulfate, filtered, and evaporated to dryness. The crude residue was purified on a BIOTAGE® 40M column (silica, hexane/ethyl acetate 9:1 to 3:2) to yield 2-(3-[N-Boc-aminophenyl]-amino)-4-chloro-6-cyclopentylamino-1,3,5-triazine as a light yellow solid: 5.3 g, 93%; LRMS (ESI): m/z 405 (MH+), 427 (M+Na); HPLC (method 1): 4.8 min. To a solution of this compound in THF (65 mL) at room temperature was added 4-aminophenethylamine (3.4 mL, 26 mmol), followed by triethylamine (6.8 mL, 39 mmol). After 20 h at 60° C. in a sealed tube, the solution was concentrated under reduced pressure. The crude residue was purified on a BIOTAGE® 40M column (silica, hexane/ethylacetate 9:1 to 1:4) to yield 2-(2-[4-aminophenyl]ethylamino)-4-(3-[N-Boc-aminophenyl]-amino)-6-cyclopentylamino-1,3,5-triazine as a white solid: 6.4 g, 97%; LRMS (ESI): m/z 505 (MH+), 527 (M+Na); HPLC (method 1): 2.4 min. To a solution of this triazine derivative (6.4 g, 12.7 mmol) in dichloromethane (62 mL) at 0° C. was added 4 N hydrochloric acid in 1,4-dioxane (31 mL, 123 mmol). The solution was stirred for 5 h at room temperature and then evaporated to dryness to yield compound 9 as a yellow solid. Yield of product: 6.5 g, 99%; ¹H NMR (400 MHz, CD₃OD): δ 7.69-7.06 (m, 8H), 4.38 (m, 1H), 3.73 (m, 2H), 3.00 (m, 2H), 2.03 (m, 2H), 1.78 (m, 2H), 1.64 (m, 2H); LRMS (ESI): m/z 405 (MH+), 427 (M+Na); HPLC (method 3): 5.5 min.

### Example 3

### Compound 1

The above compound was prepared as in Example 2. White solid; ¹H NMR (400 MHz, CD₃OD): 7.80 (m, 2H), 7.61-7.42 (m, 3H), 7.35 (m, 2H), 7.22-7.14 (s, 1H), 3.80-3.65 (m, 2H), 3.60 (m, 4H), 3.00 (m, 2H), 1.70-1.60 (m, 2H), 1.58-1.50 (m, 2H), 1.40 (m, 4H); LRMS (ESI): m/z 437 (MH+); HPLC (method 2): 2.1 min.

### Example 4

### Compound 2

The above compound was prepared as in Example 2. Pale yellow solid; ¹H NMR (400 MHz, CD₃OD): d 7.79 (m, 1H), 7.56 (m, 4H), 7.42 (m, 2H), 7.18 (m, 1H), 4.73 (d, 2H, J=12 Hz), 3.55 (m, 1H), 3.54 (t, 3H, J=7 Hz), 3.41 (t, 1H, J=6.5 Hz), 1.67 (m, 2H), 1.67 (m, 2H), 1.53 (m, 2H), 1.42 (m, 4H), 1.34 (m, 2H); LRMS (ESI): m/z 423 (MH+), 445 (M+Na), 696 (M−NH₂); HPLC (method 2): 2.8 min.

### Example 5

### Compound 3

The above compound was prepared as in Example 2. Pale yellow solid; ¹H NMR (400 MHz, D₂O): δ 7.1-7.6 (m, 8H), 3.52-3.66 (m, 2H), 3.4-3.5 (m, 2H), 3.18-3.34 (m, 2H), 2.76-2.88 (m, 2H), 1.35-1.54 (m, 4H); LRMS (ESI): m/z 410 (MH+), 432 (M+Na); HPLC (method 2): 3.7 min.

### Example 6

### Compound 4

The above compound was prepared as in Example 2. White solid; ¹H NMR (400 MHz, D₂O): δ 7.04-7.54 (m, 8H), 3.5-3.64 (m, 2H), 3.16-3.32 (m, 2H), 2.76-2.9 (m, 4H), 1.42-1.56 (m, 4H), 1.18-1.32 (m, 4H); LRMS (ESI): m/z 436 (MH+), 458 (M+Na); HPLC (method 2): 3.4 min.

### Example 7

### Compound 5

The above compound was prepared as in Example 2. Pale yellow solid; ¹H NMR (400 MHz, D₂O): δ 7.10-7.56 (m, 8H), 3.52-3.70 (m, 4H), 3.08-3.16 (m, 2H), 2.80-2.90 (m, 2H); LRMS (ESI): m/z 380 (MH+), 402 (M+Na); HPLC (method 2): 2.5 min.

### Example 8

### Compound 7

The above compound was prepared as in Example 2. Yellow solid; ¹H NMR (400 MHz, D₂O): δ 6.90-7.38 (m, 8H), 3.40-3.54 (m, 2H), 2.92-3.06 (m, 2H), 2.66-2.76 (m, 2H), 0.76-0.88 (m, 1H), 0.24-0.36 (m, 2H), 0.08 (m, 2H); LRMS (ESI): m/z 392 (MH+), 414 (M+Na); HPLC (method 2): 2.2 min.

### Example 9

### Compound 8

The above compound was prepared as in Example 1. White solid; ¹H NMR (400 MHz, CD₃OD): δ 7.99-7.19 (m, 8H), 4.54 (m, 1H), 3.76 (m, 2H), 3.01 (m, 2H), 2.38 (m, 2H), 2.16 (m, 2H), 1.84 (m, 2H); LRMS (ESI): m/z 391 (MH+), 413 (M+Na); HPLC (method 1): 1.2 min.

### Example 10

### Compound 10

The above compound was prepared as in Example 2. Pale yellow solid; ¹H NMR (400 MHz, D₂O): δ 7.0-7.4 (m, 12H), 4.52 (s, 2H), 3.4 (m, 2H), 2.6-2.9 (m, 2H); LRMS (ESI): m/z 442 (MH+), 464 (M+Na); HPLC (method 2): 3.9 min.

### Example 11

### Compound 11

The above compound was prepared as in Example 1. White solid; ¹H NMR (400 MHz, CD₃OD): δ 7.55-7.29 (m, 11H); 7.00 (t, J=4 Hz, 1H); 4.69 (s, 2H); 3.75 (dt, 1H, J=32 Hz and J=4 Hz); 3.67 (dt, 1H, J=32 Hz and J=4 Hz); 3.00 ((dt, 1H, J=32 Hz and J=4 Hz); 2.92 (dt, 1H, J=32 Hz and J=4 Hz); ¹⁹F NMR (376.5 MHz, CD₃OD): δ −77.9, ¹⁹F NMR (376.5 MHz, CD₃OD, coaxial insert trifluorotoluene): 2 TFA; LRMS (ESI) m/z 442 (MH+); HPLC (method 2): 2.2 min.

### Example 12

### Compound 12

The above compound was prepared as in Example 2. Yellow solid; ¹H NMR (400 MHz, CD₃OD): δ 7.83 (s, 1H), 7.75 (d, 2H), 7.45-7.56 (m, 6H), 7.21-7.35 (m, 1H), 7.17 (t, 2H), 4.80 (s, 2H); LRMS (ESI): m/Z 414 (MH+), 436 (M+Na); HPLC (method 1): 4.2 min.

### Example 13

### Compound 13

The above compound was prepared as in Example 2. Yellow solid; ¹H NMR (400 MHz, CD₃OD): δ 7.77 (s, 3H), 7.48-7.61 (m, 5H), 7.42 (d, 2H), 7.20 (t, 2H), 4.78 (s, 2H); LRMS (ESI): m/z 414 (MH+); HPLC (method 1): 3.9 min.

### Example 14

### Compound 14

The above compound was prepared as in Example 1. White solid; ¹H NMR (300 MHz, CD₃OD): δ 7.80 (m, 2H), 7.53 (m, 1H), 7.33 (m, 1H), 7.19 (m, 2H), 7.12 (m, 1H), 6.84 (dd, 1H, J=15 Hz and J=7 Hz), 3.81 (m, 5H), 3.26 (t, 2H, J=6 Hz); LRMS (ESI) m/z 367 (MH+), 350 (M−NH₂); HPLC (method 2): 2.8 min.

### Example 15

### Compound 15

The above compound was prepared as in Example 1. Pale yellow solid; ¹H NMR (400 MHz, CD₃OD): δ 7.79 (m, 2H), 7.43 (m, 3H), 7.17 (m, 1H), 6.99 (m, 2H), 3.83 (m, 5H), 3.29 (m, 2H); LRMS (ESI) m/z 367 (MH+), 350 (M−NH₂); HPLC (method 2): 2.7 min.

### Example 16

### Compound 16

The above compound was prepared as in Example 1. Pale yellow solid; ¹H NMR (400 MHz, CD₃OD): δ 7.99-7.15 (m, 8H), 4.73 (m, 2H); LRMS (ESI) m/z 323 (MH+), 345 (M+Na); HPLC (method 2): 2.8 min.

### Example 17

### Compound 17

The above compound was prepared as in Example 1. Yellow solid; ¹H NMR (400 MHz, CD₃OD): δ 7.78-7.36 (m, 7H), 7.08 (d, 1H, J=7.6 Hz), 4.69 (m, 2H); LRMS (ESI) m/z 323 (MH+), 345 (M+Na); HPLC (method 3): 2.4 min.

### Example 18

### Compound 18

The above compound was prepared as in Example 1. White solid; ¹H NMR (400 MHz, CD₃OD): δ 7.84-7.38 (m, 7H), 7.15 (d, 1H, J=8.2 Hz), 4.70 (m, 2H); LRMS (ESI) m/z 323 (MH+); HPLC (method 3): 4.2 min.

### Example 19

### Compound 19

The above compound was prepared as in Example 2. Pale yellow solid; ¹H NMR (400 MHz, CD₃OD): δ 7.92-7.75 (m, 4H), 7.53 (t, 2H), 7.17 (d, 2H), 3.56-3.51 (m, 4H), 1.75-1.65 (m, 2H), 1.59-1.51 (m, 2H), 1.48-1.35 (m, 4H); LRMS (ESI) m/z 409 (MH+); HPLC (method 3): 5.4 min.

### Example 20

### Compound 20

The above compound was prepared as in Example 2. White solid; ¹H NMR (400 MHz, CD₃OD): δ 7.76-7.88 (m, 4H), 7.47-7.55 (m, 4H), 7.34 (d, 2H), 7.17 (d, 2H), 3.80 (t, 2H), 3.05 (t, 2H); LRMS (ESI) m/Z 428 (MH+), 450 (M+Na), HPLC (method 1): 4.0 min.

### Example 21

### Compound 21

The above compound was prepared as in Example 2. Yellow solid; ¹H NMR (400 MHz, CD₃OD): δ 8.24-7.83 (m, 2H), 7.78 (d, 2H), 7.55 (t, 2H), 7.20 (t, 2H), 3.85 (t, 2H), 3.28 (t, 2H); LRMS (ESI) m/z 335 (MH+), 352 (M+Na); HPLC (method 3): 2.6 min.

### Example 22

### Compound 22

The above compound was prepared as in Example 1. White solid; ¹H NMR (400 MHz, CD₃OD): δ 7.74 (m, 2H), 7.49 (t, 1H, J=8 Hz), 7.41-7.21 (m, 1H), 7.13 (m, 3H), 6.89-6.79 (m, 1H), 3.82 (s, 3H), 3.76 (t, 2H, J=6 Hz), 3.63 (m, 2H); LRMS (ESI) m/z 368 (MH+), 390 (M+Na); HPLC (method 2): 3.1 min.

### Example 23

### Compound 23

The above compound was prepared as in Example 1. Pale yellow solid; ¹H NMR (400 MHz, CD₃OD): δ 7.59 (m, 2H); 7.44 (m, 3H); 6.99 (m, 3H); 3.82 (s, 3H); 3.74 (m, 2H); 3.60 (m, 2H); ¹⁹F NMR (376.5 MHz, CD₃OD): δ −77.7; ¹⁹F NMR (376.5 MHz, CD₃OD, coaxial insert trifluorotoluene): 2 TFA; LRMS (ESI) m/z 368 (MH+), 390 (M+Na); HPLC (method 1): 1.5 min.

### Example 24

### Compound 24

The above compound was prepared as in Example 1. Off-white solid; ¹H NMR (400 MHz, CD₃OD): δ 7.77 (m, 2H), 7.51 (t, 1H, J=9 Hz), 7.31 (m, 1H), 7.14 (m, 3H), 6.82 (m, 1H), 3.81 (s, 3H), 3.37 (d, 2H, J=8 Hz), 1.15 (m, 1H); 0.58 (ddd, 2H, J=12 Hz and J=5 Hz and J=2 Hz), 0.32 (dd, 2H, J=10 Hz and J=5 Hz); LRMS (ESI) m/z 378 (MH+); HPLC (method 2): 4.5 min.

### Example 25

### Compound 25

The above compound was prepared as in Example 1. Off-white solid; ¹H NMR (400 MHz, CD₃OD): δ 7.95-7.75 (m, 2H), 7.60-7.35 (m, 3H), 7.18 (m, 1H), 7.10-6.93 (m, 2H), 3.83 (s, 3H), 3.36 (m, 2H), 1.13 (m, 1H), 0.58 (m, 2H), 0.31 (m, 2H); LRMS (ESI) m/z 378 (MH+), 400 (M+Na); HPLC (method 1): 2.2 min.

### Example 26

### Compound 26

The above compound was prepared as in Example 1. White solid; ¹H NMR (400 MHz, CD₃OD): δ 7.99-7.83 (m, 1H), 7.59-7.39 (m, 4H), 7.18 (m, 1H), 6.98 (d, 2H, J=4 Hz), 3.82 (s, 3H), 1.37 (m, 1H), 0.97 (m, 2H), 0.75 (m, 2H); LRMS (ESI) m/z 364 (MH+), 386 (M+Na); HPLC (method 1): 1.9 min.

### Example 27

### Compound 27

The above compound was prepared as in Example 1. Off-white solid; ¹H NMR (400 MHz, CD₃OD): δ 7.83 (m, 1H), 7.56-7.37 (m, 4H), 7.15 (d, 1H, J=4 Hz), 7.00 (m, 2H), 3.83 (s, 3H); LRMS (ESI) m/z 324 (MH+), 346 (M+Na); HPLC (method 2): 2.4 min.

### Example 28

### Compound 28

The above compound was prepared as in Example 1. White solid; ¹H NMR (400 MHz, CD₃OD): δ 7.72 (m, 2H), 7.57 (m, 2H), 7.51 (m, 1H), 7.16 (m, 3H), 3.76 (t, 2H, J=8 Hz), 3.61 (dt, 2H, J=16 Hz and J=8 Hz); LRMS (ESI) m/z 356 (MH+), 378 (M+Na); HPLC (method 1): 1.6 min.

### Example 29

### Compound 29

The above compound was prepared as in Example 1. Yellow solid; ¹H NMR (400 MHz, CD₃OD): δ 7.87-7.01 (m, 8H), 4.67 (d, 2H, J=14 Hz), 3.74 (m, 2H), 3.60 (m, 2H); ¹⁹F NMR (376.5 MHz, CD₃OD): 6-115; LRMS (ESI) m/z 370 (MH+); HPLC (method 2): 3.2 min.

### Example 30

### Compound 30

The above compound was prepared as in Example 2. Yellow solid; ¹H NMR (400 MHz, CD₃OD): δ 7.73-6.88 (m, 8H), 3.46 (m, 2H), 3.04 (m, 2H), 2.74 (t, 2H, J=6 Hz), 0.83 (m, 1H), 0.27 (d, 2H, J=7 Hz), 0.02 (d, 2H, J=5 Hz); LRMS (ESI) m/z 391 (MH+), 413 (M+Na); HPLC (method 2): 2.5 min.

### Example 31

### Compound 31

The above compound was prepared as in Example 1. Light yellow solid; ¹H NMR (400 MHZ, CD₃OD): δ 7.41 (m, 4H), 7.15 (t, 2H, J=8 Hz), 6.76 (d, 2H, J=7 Hz), 3.07 (d, 2H, J=7 Hz), 0.85 (m, 1H), 0.27 (ddd, 2H, J=12 Hz and J=6 Hz and J=2 Hz), 0.02 (dd, 2H, J=9 Hz and J=5 Hz); ¹⁹F NMR (376.5 MHz, CD₃OD): δ −77.9, ¹⁹F NMR (376.5 MHz, CD₃OD, coaxial insert trifluorotoluene): 2 TFA; LRMS (ESI) m/z 363 (MH+), 385 (M+Na); HPLC (method 2): 2.7 min.

### Example 32

### Compound 32

The above compound was prepared as in Example 1. Off-white solid; ¹H NMR (400 MHz, CD₃OD): δ 8.03 (s, 1H), 7.86 (s, 3H), 7.53 (d, J=8 Hz, 2H), 7.17 (s, 2H); LRMS (ESI) m/z 349 (MH+); HPLC (method 3): 4.5 min.

### Example 33

### Compound 33

The above compound was prepared as in Example 1. Yellow solid; ¹H NMR (400 MHZ, CD₃OD): δ 7.80 (m, 4H), 7.52 (t, 2H, J=8 Hz), 7.16 (dd, 2H, J=8 Hz and J=2 Hz); LRMS (ESI) m/z 309 (MH+); HPLC (method 4): 2.1 min.

### Example 34

### Compound 34

The above compound was prepared as in Example 1. White solid; ¹H NMR (400 MHz, CD₃OD): δ 7.92-7.85 (m, 2H), 7.74 (d, J=7 Hz, 2H), 7.51 (t, J=8 Hz, 2H), 7.13 (dd, J=8 Hz and J=1.4 Hz, 2H), 3.77 (t, J=6 Hz, 2H), 3.64 (t, J=6 Hz, 2H); LRMS (ESI) m/z 353 (MH+); HPLC (method 3): 3.5 min.

### Example 35

### Compound 35

The above compound was prepared as in Example 1. Yellow solid; ¹H NMR (400 MHz, CD₃OD): δ 8.44 (s, 1H), 7.93 (s, 1H), 7.82 (d, J=7 Hz, 1H), 7.58-7.41 (m, 3H), 7.19 (d, J=8 Hz, 1H), 7.15 (d, J=6 Hz, 1H), 4.25-4.18 (m, 2H), 3.81-3.66 (m, 6H), 3.38 (s, 3H); LRMS (ESI) m/z 487 (MH+); HPLC (method 3): 3.6 min.

### Example 36

### Compound 36

The above compound was prepared as in Example 1. Yellow solid; ¹H NMR (400 MHz, CD₃OD): δ 8.08 (m, 1H), 7.87 (m, 2H), 7.77 (d, 1H, J=7.2 Hz), 7.54 (t, 2H, J=8.2 Hz), 7.20 (d, 2H, J=7.8 Hz), 3.55 (t, 2H, J=7.0 Hz), 2.93 (t, 2H, J=7.8 Hz), 1.71 (m, 4H), 1.49 (m, 4H); LRMS (ESI): m/z 408 (MH+); HPLC (method 2): 1.3 min.

### Example 37

### Compound 37

The above compound was prepared as in Example 1. White solid; ¹H NMR (400 MHz, CD₃OD): δ 7.88 (m, 1H), 7.72 (m, 1H), 7.54 (d, 1H, J=8.0 Hz), 7.39 (m, 1H), 7.13 (m, 3H), 6.99 (m, 1H), 3.74 (m, 2H), 3.58 (m, 2H); ¹⁹F NMR (376.5 MHz, CD₃OD): 6-127; LRMS (ESI): m/z 370 (MH+); HPLC (method 3): 5.7 min.

### Example 38

### Compound 38

The above compound was prepared as in Example 2. White-brown solid; ¹H NMR (400 MHz, CD₃OD): δ 7.72-6.85 (m, 8H), 3.50 (m, 2H), 3.03 (m, 2H), 2.77 (m, 2H), 0.81 (m, 1H), 0.25 (m, 2H), 0.02 (m, 2H); LRMS (ESI): m/z 391 (MH+), 413 (M+Na); HPLC (method 4): 4.1 min.

### Example 39

### Compound 39

The above compound was prepared as in Example 1. White solid; ¹H NMR (400 MHz, CD₃OD): δ 8.01-7.83 (m, 2H), 7.82-7.70 (m, 2H), 7.53 (t, 2H, J=8.1 Hz), 7.17 (d, 2H, J=7.4 Hz), 3.63 (dd, 2H, J=6.1, 12.3 Hz), 3.55 (d, 2H, J=7.0 Hz), 1.78 (m, 2H), 1.64 (m, 2H); LRMS (ESI): m/z 381 (MH+), 403 (M+Na); HPLC (method 3): 4.2 min.

### Example 40

### Compound 40

The above compound was prepared as in Example 1. Off-white solid; ¹H NMR (400 MHz, CD₃OD): δ 7.44 (m, 2H), 7.29 (m, 2H), 7.20 (t, 1H, J=8.0 Hz), 7.08 (dd, 2H, J=20.0, 8.0 Hz), 6.86 (d, 1H, J=8.0 Hz), 3.05 (m, 2H), 0.83 (m, 1H), 0.27 (dd, 2H, J=13.0, 6.0 Hz), 0.02 (dd, 2H, J=10.0, 5.0 Hz); LRMS (ESI): m/z 391 (MH+), 413 (M+Na); HPLC (Method 1): 2.7 min.

### Example 41

### Compound 41

The above compound was prepared as in Example 1. White solid; ¹H NMR (400 MHz, CD₃OD): δ 7.82-7.46 (m, 4H), 7.45-7.28 (m, 2H), 7.27-7.13 (m, 1H), 7.03-6.85 (m, 1H), 3.77 (t, 2H, J=5.4 Hz), 3.69-3.53 (m, 2H); LRMS (ESI): m/z 356 (MH+), 378 (M+Na); HPLC (method 1): 1.7 min.

### Example 42

### Compound 42

The above compound was prepared as in Example 2. White solid; ¹H NMR (400 MHz, CD₃OD): δ 7.66 (d, 2H, J=8.0 Hz), 7.48 (m, 1H), 7.40 (m, 2H), 7.14 (m, 1H), 7.08 (m, 2H), 4.66 (s, 2H), 3.75 (t, 2H, J=5.0 Hz), 3.62 (m, 2H); ¹⁹F NMR (376.5 MHz, CD₃OD): δ −117; LRMS (ESI): m/z 370 (MH+); HPLC (method 1): 2.7 min.

### Example 43

### Compound 43

The above compound was prepared as in Example 2. White solid; ¹H NMR (400 MHz, CD₃OD): δ 7.60 (m, 1H), 7.38 (m, 4H), 7.13 (m, 2H), 6.96 (m, 1H), 4.70 (m, 2H), 3.74 (m, 2H), 3.59 (m, 2H); ¹⁹F NMR (376.5 MHz, CD₃OD): δ −121; LRMS (ESI): m/z 370 (MH+); HPLC (method 3): 5.6 min.

### Example 44

### Compound 44

The above compound was prepared as in Example 2. White solid; ¹H NMR (400 MHZ, CD₃OD): δ 7.72 (m, 2H), 7.61 (m, 2H), 7.52 (t, 1H, J=7.2 Hz), 7.39 (m, 2H), 7.18 (d, 1H, J=7.0 Hz), 3.76 (t, 2H, J=5.2 Hz),; 3.63 (m, 2H); LRMS (ESI) m/z 372 (MH+), 394 (M+Na); HPLC (Method 1): 1.9 min.

### Example 45

### Compound 45

The above compound was prepared as in Example 2. Light yellow solid; ¹H NMR (400 MHZ, CD₃OD): δ 7.81 (d, 1H, J=8.4 Hz), 7.70 (s, 1H), 7.26 (m, 4H), 7.17 (d, 2H, J=7.4 Hz), 7.08 (ddd, 1H, J=7.6, 1.7, 0.6 Hz), 6.74 (dd, 1H, J=8.1, 1.9 Hz), 3.76 (s, 6H); ¹⁹F NMR (376.5 MHz, CD₃OD, coaxial insert trifluorotoluene): 3 TFA; LRMS (ESI) m/z 430 (MH+), 452 (M+Na); HPLC (Method 1): 3.0 min.

### Example 46

### Ability of Compounds to Mimic Protein A as Determined by Competitive Protein A Binding ELISA

As described above, this assay evaluates the ability of the exemplified compounds to mimic protein A. Such compounds can bind to the Fc portion of human IgG as ascertained by the inhibition of binding of protein A to human IgG. The competitive protein A binding ELISA assay was performed on a 96-well plate MAXISORP® surface to enhance the binding of protein A to the bottom of the plate. The wells were coated with 100 μL of protein A (0.8 μg) and incubated overnight at 4° C. After incubation, unbound protein A was removed by three washes with phosphate buffer saline (PBS). The plate was then incubated with 100 μL/well of a 2% solution of bovine serum albumin (BSA) for 1 h at 37° C. to block non specific protein binding. After incubation, the plate was washed three times with PBS. 50 μL of compound or protein A, diluted in PBS or PBS-20% DMSO at appropriate concentration, were added to the wells followed by addition of 50 μL of peroxidase-conjugated human IgG (HRP-IgG). After 1 h incubation at 37° C., the plate was washed three times with PBS to remove unbound HRP-IgG. Bound HRP-IgG was detected by incubation with 100 μL of 2,2′-azino-di[3-ethylbenzthiazoline sulfonate] diammonium salt crystals (ABTS) solution for 20 min in the dark at room temperature. The plate was then read at 405 nm on a EL 800, universal microplate reader (Bio-Tek). Data was analyzed in Microsoft Excel and the concentration of compound which inhibits 50% binding of protein A (IC₅₀) was calculated using Prism software.

TABLE 1
IC₅₀ (nM) of protein A mimic compounds as ascertained by ELISA.

| | IC₅₀ (nM) |
|---|---|
| Compound No. | Assay in PBS |
| 1 | 93 |
| 2 | 148 |
| 3 | 106 |
| 4 | 300 |
| 5 | 377 |
| 6 | 73 |
| 7 | 55 |
| 8 | 53 |
| 9 | 131 |
| 10 | 106 |
| 11 | 85 |
| 12 | 3 |
| 13 | 1 |
| 14 | 306 |
| 15 | 415 |
| 16 | 159 |
| 17 | 65 |
| 18 | 97 |
| 19 | 15 |
| 20 | 65 |
| 21 | 114 |
| 22 | 63 |
| 23 | 86 |
| 24 | 243 |
| 25 | 75 |
| 26 | 126 |
| 27 | 292 |
| 28 | 62 |
| 29 | 222 |
| 30 | 88 |
| 31 | 38 |
| 32 | 38 |
| 33 | 170 |
| 34 | 42 |
| 35 | 37 |
| 36 | 68 |
| 37 | 94 |
| 38 | 312 |
| 39 | 40 |
| 40 | 586 |
| 41 | 36 |
| 42 | 39 |
| 43 | 86 |
| 44 | 56 |
| 45 | 469 |
| Protein A (Control) | 187 |

### Example 47

### Effect of Compounds on Oxazolone-Induced Delayed-Type Hypersensitivity

Compounds were tested for their ability to treat oxazolone-induced delayed-type hypersensitivity (DTH) in mice. On day 0, mice were sensitized with 100 μL of oxazolone in 5% acetone. On day 0, 1 and 2, mice were treated by intravenous administration of the vehicle (control) or methotrexate (MTX; positive control) or the compound at 50 mg/kg (compound 3) or 25 mg/kg (compound 1 or 10). Mice were challenged with an application of 50 μL of oxazolone on the surface of the right ear (first challenge, day 3; second challenge, day 10). Ear thickness was measured on day 4 to day 7, and on day 11 to 14. Redness and crust formation was also observed. Mice were sacrificed on day 14. T_{DTH} (CD4) cells play an important role in regulating the intensity of the DTH response. Compounds may exert an inhibitory influence on the DTH response through its inhibition of T-cell activation and DNA, RNA and/or protein synthesis.

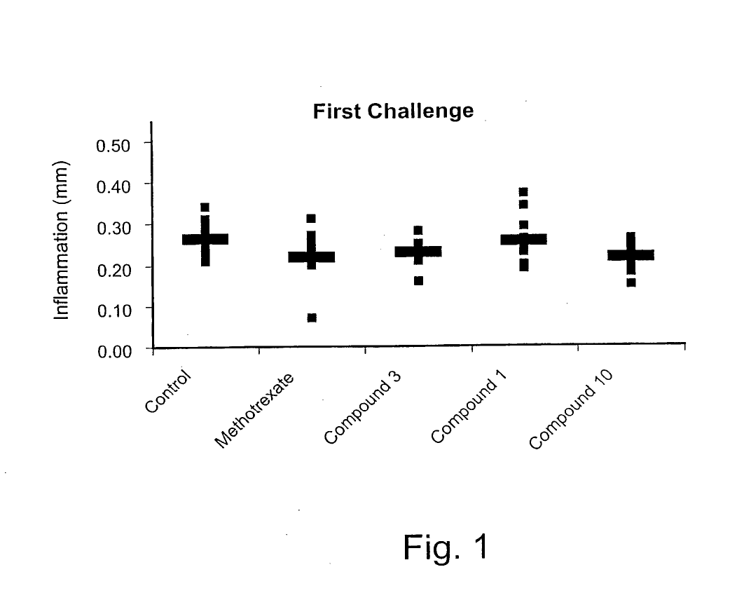

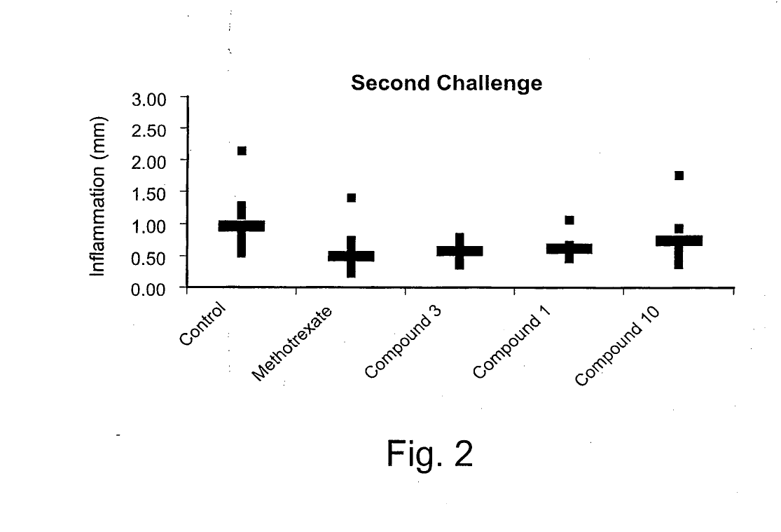

As illustrated in FIG. 1, all compounds induce a significant reduction of the inflammation as seen by lower ear thickness. Also, all compounds are equipotent to methotrexate. Compounds also reduce redness, crust formation and ear swelling. As presented in FIG. 2, compounds 1 and 3 sustain a significant inhibition of the inflammation after the second challenge of oxazolone.

Additionally, Table 2 summarizes the effect of compounds that mimic protein A on DTH. Compounds were administered intravenously unless specified. These compounds induce a significant inhibition of inflammation as demonstrated by the diminution of ear thickness. The inhibition of inflammation is observed after challenge 1 or after challenge 2 or both. Furthermore, oral activity was also observed for compounds 1, 3, 5 and 10.

TABLE 2
Effect of compounds 1, 2, 3, 5, 9, 10, 16, 19, 20, 28, 29, 31 and 35
on DTH.

Activity (ear thickness inhibition)

| Compounds | First Challenge | Second Challenge |
|---|---|---|
| 1 | No effect | ↓ (p = 0.03) |
| | ↓ per os (p = 0.003) | ↓ (n.s.)* |
| 2 | ↓ (p = 0.04) | No effect |
| 3 | ↓ (p = 0.03) | ↓ (p = 0.02) |
| | ↓ per os (p = 0.03) | ↓ (n.s.) |
| 5 | ↓ per os (p = 0.008) | ↓ (n.s.) |
| 9 | ↓ (0.01) | No effect |
| 10 | ↓ p = 0.006 | No effect |
| | No effect per os | ↓ (p = 0.04) |
| 16 | No effect | No effect |
| 19 | ↓ (n.s.) | No effect |
| 20 | ↓ (p = 0.03) | ↓ (p = 0.02) |
| 28 | ↓ (n.s.) | ↓ (p = 0.04) |
| 29 | ↓ (p = 0.004) | ↓ (p = 0.01) |
| 31 | ↓ (p = 0.002) | ↓ (n.s.) |
| 35 | ↓ (p = 0.003) | No effect |

*(n.s.) = non significant effect

### Example 48

### Effect of Compounds on Freund's Adjuvant-Induced Arthritis (AIA)

AIA was induced in female Lewis rats by the injection of lyophilized Mycobacterium butyricum suspended in mineral oil into the footpad. The development of arthritis was monitored over a 3 weeks period post-adjuvant injection. Inflammation peaks at day 3 following the adjuvant administration. Immune activation appears around day 14. Compounds were injected at different doses at day −3, −2 and −1 pre-adjuvant injection and at day 10, 11 and 12 post-adjuvant injection. Body weight was recorded. The arthritis index, which is a measure of inflammation (oedema), redness and stiffness of the articulations, was used to monitor the development of the disease. The degree of arthritis was determined by measuring two perpendicular diameters of the ankles in the mediolateral and dorsoventral planes using a caliper. Joint circumference in millimeters is then calculated using a geometric formula. Both the incidence and severity of the arthritis was evaluated. Incidence is defined as the number of rats with clinical evidence of joint inflammation during the study period.

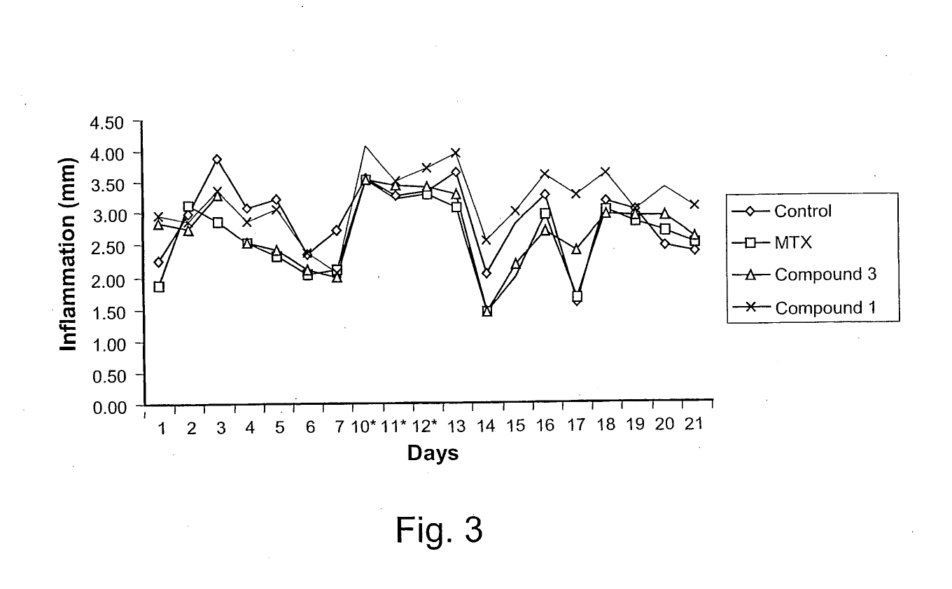

As illustrated in FIG. 3, 100% of the animals rapidly developed a synovitis. Inflammation reaches its maximum at day 3 postimmunization. A significant reduction (up to 30%) in the severity of arthritis (inflammatory index) was observed by intravenous injection of methotrexate (positive control) on day 3, 4, 5, 7, 13 and 15; compound 1 on day 3 and 7; and compound 3 on day 3, 4, 5, 7 and 15.

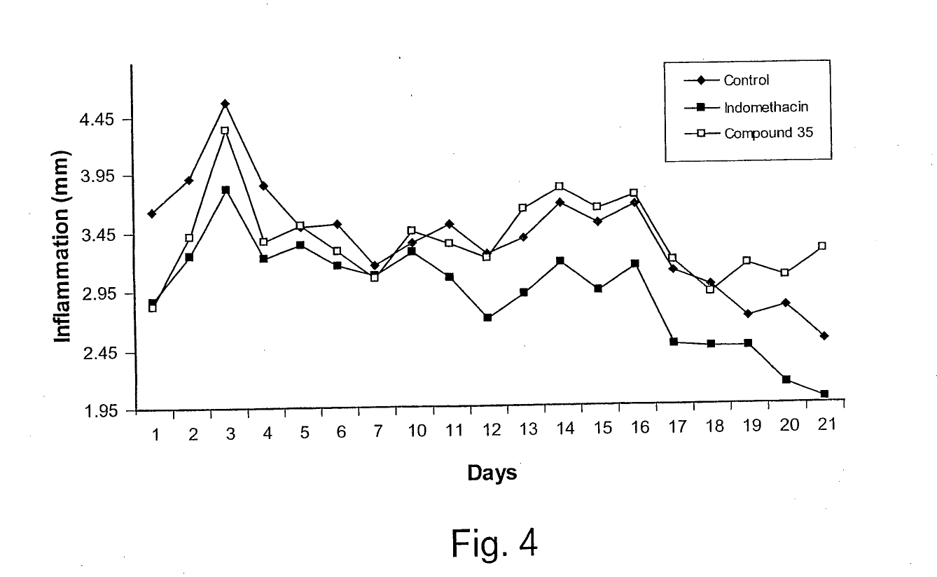

As illustrated in FIG. 4, a significant reduction (up to 25%) in the severity of arthritis (inflammatory index) was observed by oral administration of indomethacin (positive control) on day 1, 2, 3, 4, 11, 12, 13, 15, 16, 17 and 20; and compound 35 on day 1 and 4.

### Example 49

### Use of Compounds to Bind and Purify Immunoglobulins. Covalent Attachment of Compounds to an Insoluble Support Material

As noted above, exemplified compounds may be used as affinity agents to bind antibody and subsequently isolate and purify the antibody from mixtures. Moreover, exemplified compounds may be used as affinity agents to bind a monoclonal antibody and subsequently isolate and purify the antibody from mixtures containing a non-ionic detergent such as PLURONIC® F-68. Such purification is conveniently accomplished when the compound is first covalently linked, either directly or by means of a linker, to an insoluble support material. Various methodologies may be used to achieve this covalent link, including, but not limited to, those detailed below. The packed gels (200 μL in a spin column) were equilibrated in 20 mM PBS (pH=7). In this format, the immobilized compounds may be used for immobilization of antibody and subsequent purification.

### Example 49-1

### Compound 5 Directly-Linked to SEPHAROSE® 6B: (Compound 5)-SEPHAROSE® 6B

A solution of compound 5 (1.97 g, 3.75 mmol) in water (50 mL) was treated with epoxide activated cross-linked (with epichlorohydrin) SEPHAROSE® 6B beads (50 g), and the slurry was adjusted to pH=5.5 with 10 M NaOH. The reaction was shaken on a rocker plate for 24 h. The slurry was adjusted to pH=1.0-2.0 with 1N HCl, and the reaction was shaken for a further 25 min. The beads were filtered, washed with 0.1N HCl (3×100 mL) and with water (5×100 mL), then resuspended in water (50 mL) and treated with 10 M NaOH (10 mL). The reaction was shaken on a rocker plate for 24 h. The beads were filtered, and washed with water (7×100 mL) until the pH of the filtrate was neutral, to yield a pink gel. A sample was freeze-dried for elemental analysis: C, 50.729%; H, 6.727%; N, 6.603%. Based on nine atoms of nitrogen per molecule of compound 5, this corresponds to a loading of 524 μmol/g freeze dried gel.

### Example 49-2

### (Compound 5)-SEPHAROSE® 6B

Alternatively, compound 5 was covalently linked according to Example 49-1, but with the use of a solution of compound 5 in 50% aqueous acetone, and adjusting the pH to 10-11, to yield a pale pink gel (466 μmol/g freeze dried gel).

### Example 49-3

### Compound 5 Linked to SEPHAROSE® 6B Via a 6-aminohexanoic Acid Linker: (Compound 5)-6AHA-SEPHAROSE® 6B

85 g of epoxide activated cross-linked (with epichlorohydrin) SEPHAROSE® 6B beads were treated with a solution of 6-aminohexanoic acid (6.80 g, 52 mmol) in water (85 mL) and the slurry was adjusted to pH=12 with 10 M NaOH. The reaction was shaken on a rocker plate overnight. The beads were filtered, washed with water (10×85 mL), resuspended in water (85 mL), and treated with 10 M NaOH (17 mL). The reaction was shaken on a rocker plate for 28 h. The beads were filtered, washed with water (10×170 mL) until the pH of the filtrate was neutral, and a sample of the gel was freeze-dried for elemental analysis: C, 47.854%; H, 7.024%; N, 0.856%. Based on one atom of nitrogen per molecule of 6-aminohexanoic acid, this corresponds to a loading of 611 mmol/g freeze dried gel. The settled gel (35 g) was treated with a solution of compound 5 (1.38 g, 2.63 mmol) in water (30 mL), and a solution of 1-[3-(dimethylamino)propyl]-3-ethylcarbodiimide hydrochloride (3.50 g, 18.3 mmol) in water (5 mL). The slurry was measured as pH=4.0-4.5, needed no adjustment. The reaction was then shaken on a rocker plate overnight. The slurry was adjusted to pH=1.0-2.0 with 1N HCl and the reaction was shaken for a further 5-25 min. The beads were filtered, washed with 0.1M HCl (3×70 mL) and water (10×70 mL) to yield an off-white gel. A sample was freeze-dried for elemental analysis: C, 46.993%; H, 6.815%; N, 4.967%. Based on nine atoms of nitrogen per molecule of compound 5, this corresponds to a loading of 326 μmol/g freeze dried gel.

### Example 49-4

### (Compound 5)-6AHA-SEPHAROSE® 6B

Alternatively, compound 5 was covalently linked according to Example 49-3, but with substitution of the coupling agent N-[4,6-dimethoxy-1,3,5-triazin-2-yl]-N-methyl-morpholinium chloride for 1-[3-(dimethylamino)propyl]-3-ethylcarbodiimide hydrochloride, to yield an off-white gel (275 μmol/g freeze dried gel).

### Example 49-5

### (Compound 5)-6AHA-SEPHAROSE® 6B

Alternatively, compound 5 was covalently linked according to Example 49-3, but with a two step coupling procedure (addition of N-hydroxysuccinimide in place of compound 5; shaking for 2 hours; filtration and washing with 10 volumes of water; suspension in a pH=5.0 aqueous solution of compound 5; and shaking overnight), to yield an off-white gel (294 μmol/g freeze dried gel).

### Examples 49-6 to 49-23

### Other Ligands and Support Materials

The same procedures described above were employed to covalently link other compounds of this invention to insoluble support materials, substituting the support material and/or linker reported in the above examples as required, as summarized in Table 3.

TABLE 3
Summary of other ligands bound to support materials.

| Example | Compound | Support | Linker* | Method | Loading** |
|---|---|---|---|---|---|
| 49-6 | 5 | PURABEAD ® 6 | none | 49-1 | 539 |
| 49-7 | 5 | PURABEAD ® 6 | none | 49-2 | 493 |
| 49-8 | 5 | SEPHAROSE ® 6- | none | 49-1 | 363 |
| | | CL | | | |
| 49-9 | 5 | PURABEAD ® 6XL | none | 49-1 | 403 |
| 49-10 | 5 | PURABEAD ® 6XL | none | 49-2 | 408 |
| 49-11 | 4 | SEPHAROSE ® 6B | none | 49-1 | 435 |
| 49-12 | 4 | SEPHAROSE ® 6B | none | 49-2 | 312 |
| 49-13 | 5 | PURABEAD ® 6 | 6AHA | 49-3 | 394 |
| 49-14 | 5 | SEPHAROSE ® 6-CL | 6AHA | 49-3 | 296 |
| 49-15 | 5 | PURABEAD ® 6XL | 6AHA | 49-3 | 335 |
| 49-16 | 5 | SEPHAROSE ® 6B | L-Asp | 49-3 | 441 |
| 49-17 | 5 | SEPHAROSE ® 6B | L-Asp | 49-4 | 302 |
| 49-18 | 5 | SEPHAROSE ® 6B | β-Glu | 49-3 | 433 |
| 49-19 | 5 | SEPHAROSE ® 6B | β-Ala- | 49-3 | 299 |
| | | | 6AHA | | |
| 49-20 | 5 | SEPHAROSE ® 6B | β-Ala- | 49-5 | 309 |
| | | | 6AHA | | |
| 49-21 | 5 | SEPHAROSE ® 6B | β-Ala-β- | 49-3 | 315 |
| | | | Ala | | |
| 49-22 | 4 | SEPHAROSE ® 6B | 6AHA | 49-3 | 333 |
| 49-23 | 4 | SEPHAROSE ® 6B | 6AHA | 49-5 | 295 |

*6AHA = 6-aminohexanoic acid; L-Asp = L-aspartic acid; β-Glu = 3-aminopentanedioic acid; β-Ala = 3-aminopropionic acid. For examples where a dipeptide linker was utilised, the compound was first derivatised with one amino acid, and this derivative was then covalently linked as per the cited example.
**Expressed in μmol/g freeze dried gel.

### Example 50

### Use of Compounds to Bind and Purify Human Immunoglobulin G

This solid phase binding assay evaluates the ability of the exemplified compounds for their ability to bind, remove, and/or purify immunoglobulins. Thus, columns loaded with gels from Example 49 were treated with an excess of total human IgG (Sigma, St. Louis, USA; purified human IgG isolated from pooled normal human serum) and flow through was collected (“flow through” or non-bound fraction). The gel was washed with 5-column volumes of 20 mM sodium phosphate buffer (pH=7) plus 0.25M NaCl. Washed fractions were collected (“wash” fractions). Bound IgGs were eluted at low pH with 0.1M citric acid (pH=3); (“elution” fraction). The UV absorbance of each fraction was measured at 280 nm, and was expressed as a percentage of the UV₂₈₀ absorbance of the initial IgG solution. The results for the exemplified gels are shown in Table 4.

TABLE 4
Ability of exemplified gels to bind and elute human IgG. Values are
expressed as a percentage of the total IgG load.

| | Spin | Flow | Wash | Elution | Total Recovery |
|---|---|---|---|---|---|
| Example | Column | Through | (mL) | (mL) | (%) |
| 50-1 | 49-1 | 24.3 | 4.2 | 65.2 | 93.7 |
| 50-2 | 49-2 | 15.0/26.3 | 0.0/4.0 | 66.9/60.6 | 81.9/90.8 |
| 50-3 | 49-3 | 19.3 | 1.9 | 64.7 | 85.9 |
| 50-4 | 49-4 | 51.9 | 0.0 | 35.9 | 87.8 |
| 50-5 | 49-5 | 54.5 | 3.6 | 34.5 | 92.6 |
| 50-6 | 49-6 | 22.2 | 2.2 | 53.4 | 77.8 |
| 50-7 | 49-7 | 26.3/30.3 | 4.1/4.1 | 53.7/54.0 | 84.1/88.4 |
| 50-8 | 49-8 | 58.4 | 3.9 | 27.0 | 89.3 |
| 50-9 | 49-9 | 65.7 | 5.3 | 23.0 | 94.0 |
| 50-10 | 49-10 | 59.3 | 4.8 | 28.1 | 92.2 |
| 50-11 | 49-11 | 46.8 | 2.6 | 44.1 | 93.5 |
| 50-12 | 49-12 | 49.1 | 2.9 | 41.5 | 93.6 |
| 50-13 | 49-13 | 20.9 | 2.2 | 59.4 | 82.5 |
| 50-14 | 49-14 | 35.6 | 3.5 | 48.5 | 87.5 |
| 50-15 | 49-15 | 55.4 | 3.9 | 32.9 | 92.2 |
| 50-16 | 49-16 | 50.0 | 0.0 | 40.7 | 90.7 |
| 50-17 | 39-17 | 63.6 | 0.0 | 15.4 | 79.0 |
| 50-18 | 49-18 | 30.1 | 0.0 | 59.1 | 89.2 |
| 50-19 | 49-19 | 16.1 | nd | 61.1 | 77.7 |
| 50-20 | 49-20 | 54.3 | 0.0 | 44.3 | 98.5 |
| 50-21 | 49-21 | 27.7 | nd | 46.0 | 73.7 |
| 50-22 | 49-22 | 28.9/29.4 | 0.0/0.0 | 49.0/44.1 | 77.9/73.5 |
| 50-23 | 49-23 | 58.2/47.9 | 0.0/0.0 | 25.0/28.2 | 83.2/76.1 |
| 50-24 | negative | 97.1 | 3.1 | 2.0 | 102.2 |
| | control* | | | | |

*1,3-Phenylenediamine covalently bound to SEPHAROSE ® 6B

### Example 51

### Use of Compounds to Bind and Purify Human Immunoglobulin G in the Presence of PLURONIC® F-68

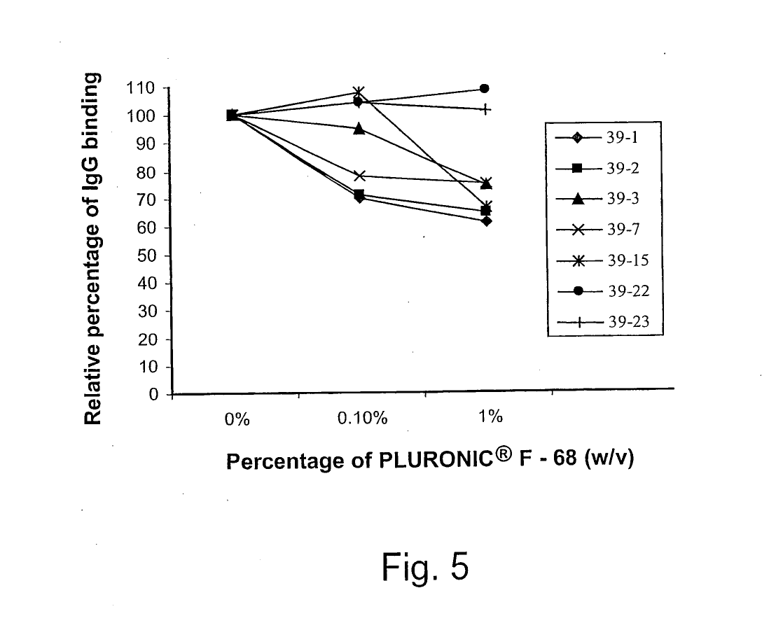

The solid phase binding assay reported in Example 50 was repeated for certain gels, using the same method as for Table 4, but with the presence of 0.1% w/v or 1.0% w/v PLURONIC® F-68 in the human IgG solution loaded onto the gels. The results are shown in Table 5 and FIG. 5.

TABLE 5
Ability of exemplified gels to bind and elute human IgG in the presence of
PLURONIC ® F-68. Values are expressed as a percentage of the total IgG load.

| | Without | | | 0.1% | | | 1.0% | | |
|---|---|---|---|---|---|---|---|---|---|
| | PLURONIC ® F-68 | | | PLURONIC ® F-68 | | | PLURONIC ® F-68 | | |
| Spin | Flow | | | Flow | | | Flow | | |
| Column | Through | Elution | Total* | Through | Elution | Total* | Through | Elution | Total* |
| 49-1 | 28.2 | 64.1 | 92.3 | 58.6 | 44.7 | 103.3 | 62.5 | 39.2 | 101.7 |
| 49-2 | 20.2 | 71.9 | 92.1 | 49.6 | 51.0 | 100.6 | 44.7 | 46.6 | 91.3 |
| 49-3 | 25.2 | 74.4 | 99.6 | 24.7 | 70.6 | 95.3 | 45.2 | 55.3 | 100.5 |
| 49-7 | 50.1 | 49.1 | 99.2 | 61.4 | 38.2 | 99.6 | 68.8 | 36.9 | 105.7 |
| 49-15 | 50.0 | 46.7 | 96.7 | 42.8 | 50.2 | 93.0 | 68.8 | 31.0 | 99.8 |
| 49-22 | 18.2 | 67.3 | 85.5 | 20.3 | 70.0 | 90.3 | 17.8 | 72.8 | 90.6 |
| 49-23 | 30.9 | 51.6 | 82.5 | 31.7 | 53.9 | 85.6 | 32.9 | 52.3 | 85.2 |

*Excluding any IgG present in the wash fraction

### Example 52

### Use of Compounds to Bind and Purify Mouse Monoclonal Antibodies from Harvested Cell Culture Fluid Containing PLURONIC® F-68

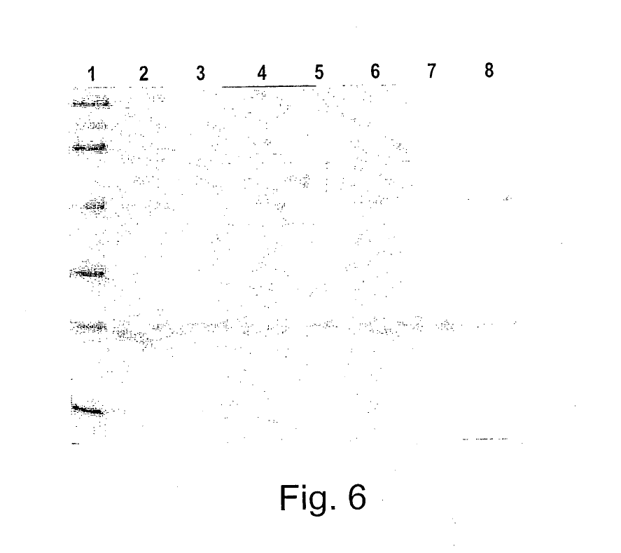

A sample of mouse monoclonal antibodies in harvested cell culture fluid containing PLURONIC® F-68 was introduced into the spin column from Example 49, such as to exceed the binding capacity of the gel, and the flow through was collected. The gel was then washed with five column volumes of 20 mM PBS (pH=7) plus 0.25 M NaCl. Wash fractions were collected. Bound monoclonal antibody was eluted at low pH with 0.1M citric acid (pH=3.0). Eluted antibody was neutralized with Tris HCl (pH=8). SDS-PAGE (12%) was performed on the collected fractions as shown in FIG. 6.

### Example 53

### Use of Compounds to Bind and Purify Rat, Mouse or Human Immunoglobulin G

The solid phase binding assay reported in Example 50 was repeated for a gel from Example 49-3, using the same method as for Table 4, in rat, mouse or human IgG solution loaded onto a gel from Example 49-3. The results are shown in Table 6. In summary, exemplified gel 49-3 binds and elutes rat, mouse or human IgG.

TABLE 6
Ability of exemplified gel from Example 39-3 to bind and elute rat, mouse
or human IgG. Values are expressed as a percentage of the total IgG load.

| | Relative percentage of IgG binding | | | |
|---|---|---|---|---|
| Gel | Fraction | Rat IgG | Mouse IgG | Human IgG |
| Example 49-3 | Flow through | 26.6 | 32.2 | 19.3 |
| | Elution pH3 | 76.1 | 63.3 | 64.7 |

### Example 54

### Use of Compounds to Bind and Purify Human IgA, IgM, IgG, IgG-Fab Fragment or IgG-Fc Fragment

The solid phase binding assay reported in Example 50 was repeated for a gel from Example 49-3, using the same method as for Table 4. The results are shown in Table 7. In summary, exemplified gel from Example 49-3 binds and elutes human IgA, IgM, IgG or IgG-Fc fragment. Weak and non-significant binding of IgG-Fab fragment was observed.

TABLE 7
Ability of exemplified gel from Example 49-3 to bind and elute human
IgA, IgM, IgG, IgG-Fab fragment or IgG-Fc fragment. Values are
expressed as a percentage of the immunoglobulin load.

| | Relative percentage of human | | | | | |
|---|---|---|---|---|---|---|
| | Immunoglobulin binding | | | | | |
| | | | | hIgG | | |
| Gel | Fraction | hIgA | hIgM | total | hIgG-Fab | hIgG-Fc |
| Example | Flow through | 46.6 | 62.9 | 19.3 | 80.3 | 21.4 |
| 49-3 | Elution pH3 | 43.5 | 42.6 | 64.7 | 14.4% | 65.4 |

### Example 55

### Use of Compounds to Bind and Purify Human IgG Subclasses

The solid phase binding assay reported in Example 50 was repeated for a gel from Example 49-3, using the same method as for Table 4. The results are shown in Table 8. In summary, exemplified gel from Example 49-3 binds and elutes all human IgG subclasses (1, 2, 3 and 4).

TABLE 8
Ability of exemplified gel from Example 49-3 to bind and elute human
IgG subclasses. Values are expressed as a percentage of the
immunoglobulin load.

| | Relative percentage of | | | | |
|---|---|---|---|---|---|
| | human IgG subclasses binding | | | | |
| Gel | Fraction | IgG1 | IgG2 | IgG3 | IgG4 |
| Example 49-3 | Flow Through | 50.5 | 10.2 | 15.0 | 11.5 |
| | Elution pH3 | 54.2 | 87.2 | 64.3 | 83.4 |

Patents, patent applications, and other publications cited herein are incorporated by reference in their entirety.

All modifications and substitutions that come within the meaning of the claims and the range of their legal equivalents are to be embraced within their scope. A claim using the transition “comprising” allows the inclusion of other elements to be within the scope of the claim; the invention is also described by such claims using the transitional phrase “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. Any of the three transitions can be used to claim the invention.

It should be understood that an element described in this specification should not be construed as a limitation of the claimed invention unless it is explicitly recited in the claims. Thus, the claims are the basis for determining the scope of legal protection granted instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of the individual elements disclosed herein are considered to be aspects of the invention; similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1-23. (canceled)

24. A compound of the following general formula: 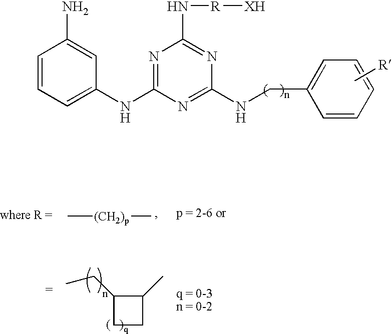
X=NH, O, S
and R′=NH₂, OCH₃, F or Cl
or where —R—XH is replaced by 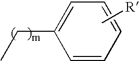
m=1-2
wherein R′, m and n are defined as above and m may or may not be equal to n or wherein —R—XH is replaced by —H.

25. The compound according to claim 1, wherein:
R=—(CH₂)ₚ—, p=2-6
X=NH, O
and R′=NH₂, OCH₃.

26. The compound according to claim 1, wherein —R—XH is replaced by 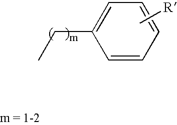
R′=NH₂ or OCH₃.

27. The compound according to claim 1, wherein R′ is meta amino, n=0 and —R—XH is replaced such that the general formula is: 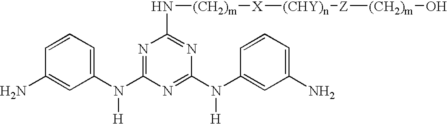
wherein m=1-2, n=2-4, X=CHY, O, S; Y=H, OH; and Z=zero, O, S.

28. The compound according to claim 1, wherein —R—XH is replaced by —H and R′=NH₂ or OCH₃.

29. A compound selected from the group consisting of:

| Compound | |
|---|---|
| No. | Structure |
| 1 | 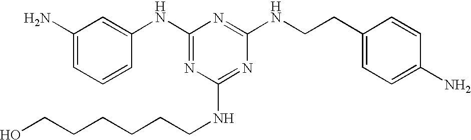 |
| 2 | 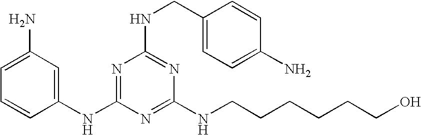 |
| 3 | 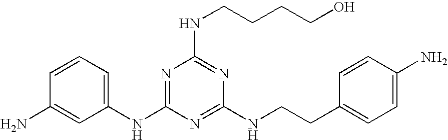 |
| 4 | 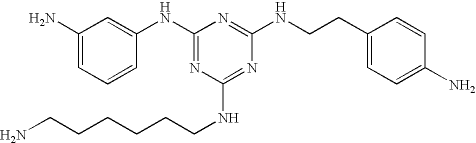 |
| 5 | 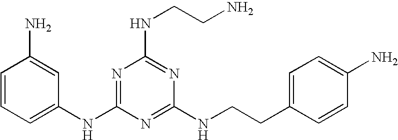 |
| 6 | 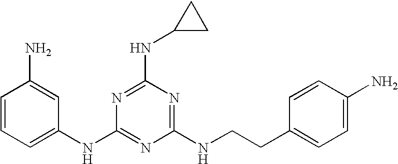 |
| 7 | 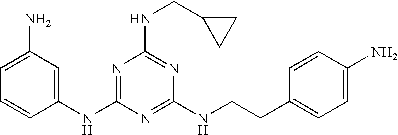 |
| 8 | 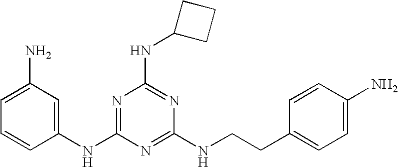 |
| 9 | 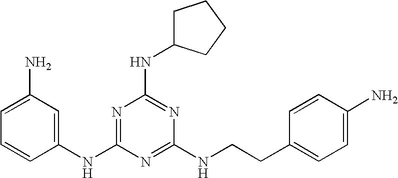 |
| 10 | 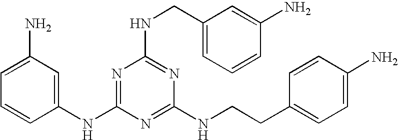 |
| 11 | 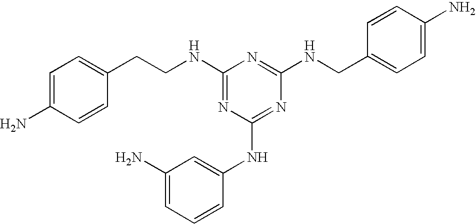 |
| 12 | 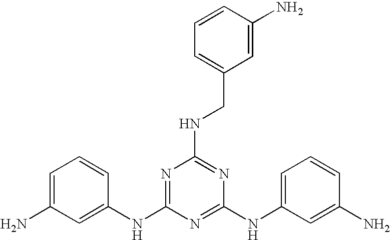 |
| 13 | 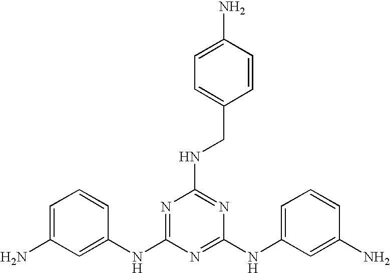 |
| 14 | 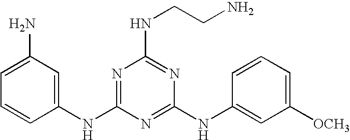 |
| 15 | 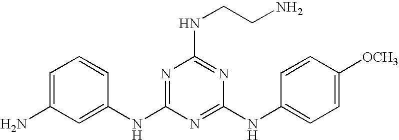 |
| 16 | 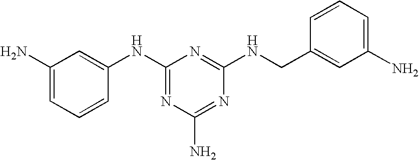 |
| 17 | 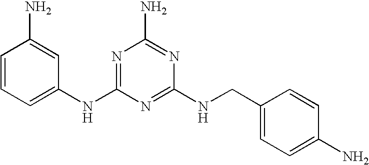 |
| 18 | 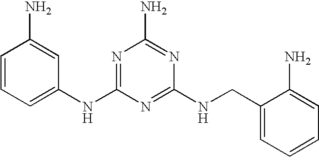 |
| 19 | 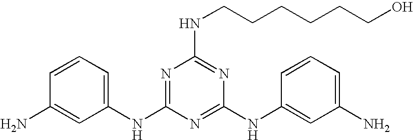 |
| 20 | 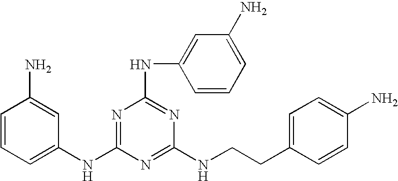 |
| 21 | 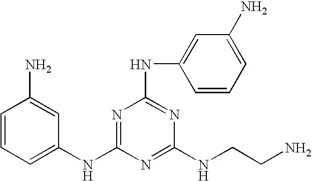 |
| 22 | 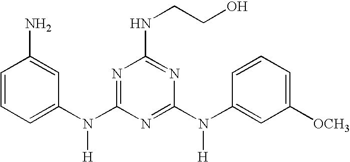 |
| 23 | 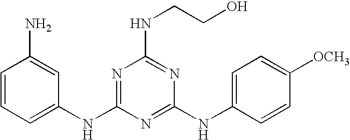 |
| 24 | 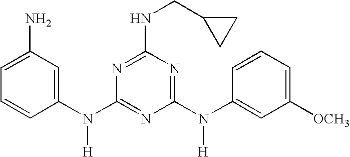 |
| 25 | 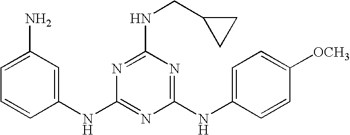 |
| 26 | 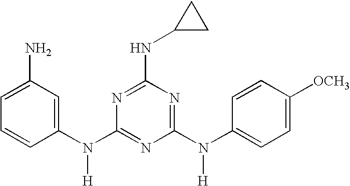 |
| 27 | 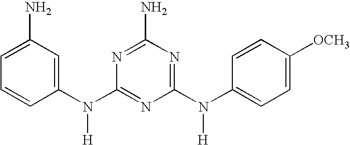 |
| 28 | 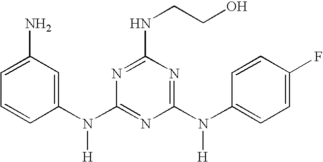 |
| 29 | 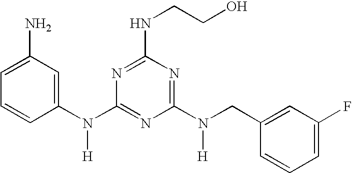 |
| 30 | 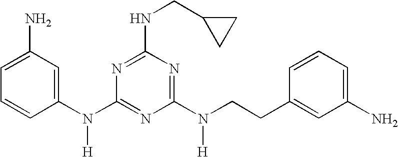 |
| 31 | 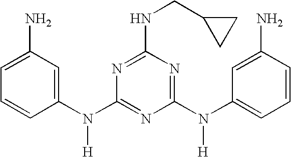 |
| 32 | 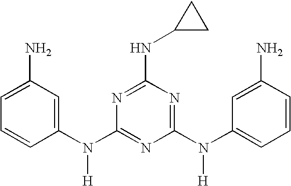 |
| 33 | 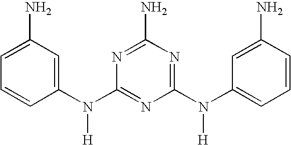 |
| 34 | 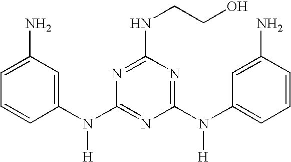 |
| 35 | 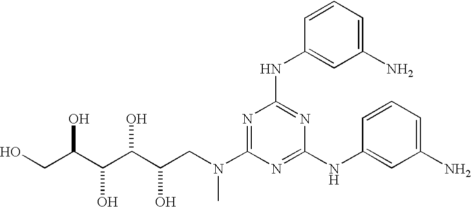 |
| 36 | 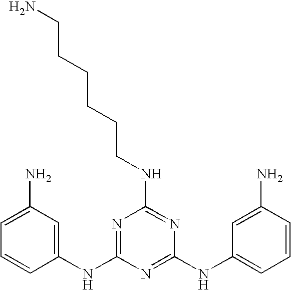 |
| 37 | 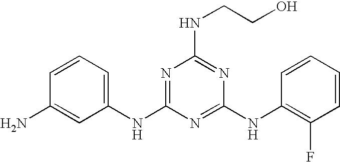 |
| 38 | 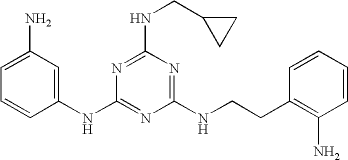 |
| 39 | 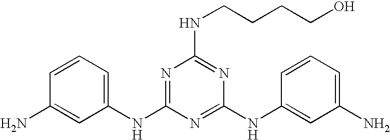 |
| 40 | 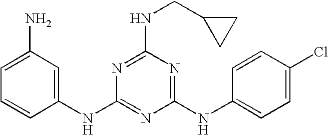 |
| 41 | 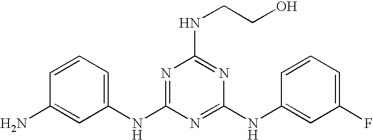 |
| 42 | 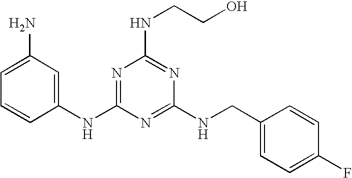 |
| 43 | 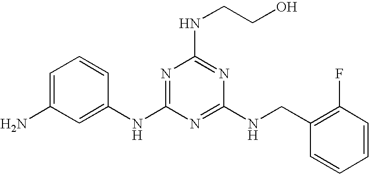 |
| 44 | 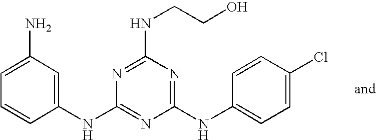 |
| 45 | 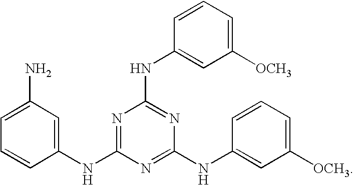 |

30. The compound according to claim 24, wherein said compound can noncovalently bind to antibodies.

31. The compound according to claim 24, wherein said compound can noncovalently bind to antibodies wherein at least two of the three substituents from the triazine scaffold are as follows:

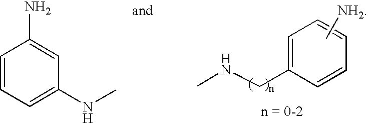

32. The compound according to claim 30, wherein said antibodies are at least of the human IgG isotype.

33. A composition comprised of at least one compound according to claim 24 and a pharmaceutically acceptable carrier.

34. The composition according to claim 33, wherein said carrier solubilizes said compound in an alcohol or polyol solvent.

35. The composition according to claim 33 further comprised of a recombinant protein which is able to bind human TNFα.

36. The composition according to claim 35, wherein said recombinant protein is anti-TNFα antibody or soluble TNFα receptor.

37. The composition according to claim 33 further comprised of methotrexate.

38. The composition according to claim 33 further comprised of an anti-inflammatory corticosteroid.

39. The composition according to claim 33 further comprised of a nonsteroidal anti-inflammatory drug.

40. A method of treating a patient with an autoimmune disease, comprising administration to said patient a therapeutically effective amount of a compound according to claim 24 or a composition according to claim 24 and a pharmaceutically acceptable carrier.

41. The method of claim 40, wherein said autoimmune disease is selected from the group consisting of systemic lupus erythematosus, immune thrombocytopenia, glomerulonephritis, vasculitis, and arthritis.

42. The method of claim 40 further comprising simultaneous administration of a therapeutically effective amount of a recombinant protein which is able to bind to human TNFα, wherein said therapeutically effective amount of recombinant protein is reduced in the presence of said compound.

43. The method of claim 40 further comprising separate administration of a therapeutically effective amount of a recombinant protein which is able to bind to human TNFα before and/or after administration of said compound, but not simultaneous administration.

44. A method of removal of human antibodies comprised of circulating blood or other physiological fluid through an apheresis column, wherein one or more compounds according to claim 24 are covalently linked either directly or with an organic linker to an insoluble support material which constitutes part of said apheresis column such that at least some free antibodies and/or antibody-antigen immune complexes are bound thereto; and returning at least some said blood or other physiological fluid, wherein at least some human antibodies have been removed therefrom, to a patient from whom said blood or other physiological fluid was obtained.

45. A method of purification of antibodies comprised of binding antibodies with one or more compounds according to claim 24 covalently linked either directly or with an organic linker to an insoluble support material such that at least some antibodies are noncovalently bound to said compounds linked to the insoluble support and purifying said antibodies.

46. The method of claim 45, wherein said antibodies to be purified are part of a mixture of proteins and non-protein material which includes a non-ionic detergent.
